(19) United States
(12) Patent Application Publication
(10) Pub. No.: US 2025/0060929 A1
(43) Pub. Date: Feb. 20, 2025

(54) ELECTRONIC DEVICE AND METHOD FOR DISPLAYING INFORMATION ON USER THROUGH EXTERNAL ELECTRONIC DEVICE

(71) Applicant: SAMSUNG ELECTRONICS CO., LTD., Suwon-si (KR)

(72) Inventors: Suntae JOO, Suwon-si (KR); Taehwan SON, Suwon-si (KR); Sunghee JUNG, Suwon-si (KR); Daesung CHO, Suwon-si (KR)

(21) Appl. No.: 18/744,341

(22) Filed: Jun. 14, 2024

Related U.S. Application Data
(63) Continuation of application No. PCT/KR2024/007806, filed on Jun. 7, 2024.

(30) Foreign Application Priority Data
Aug. 19, 2023 (KR) 10-2023-0108661
Sep. 20, 2023 (KR) 10-2023-0125963

(51) Int. Cl. G06F 3/14; G06F 3/01

(52) U.S. Cl. CPC G06F 3/1454; G06F 3/011

(57) ABSTRACT

According to an embodiment, an electronic device includes: at least one sensor configured to detect health information of a user, a communication circuit configured to perform communication with an external device, memory including one or more storage mediums storing instructions, and at least one processor comprising processing circuitry, wherein the instructions, when being executed by the at least one processor individually or collectively, cause the electronic device to: identify first level information of the health information of the user obtained based on the at least one sensor of the electronic device, based on establishing a connection with a first external electronic device, identify second level information related to the first external electronic device, and based on the first level information and the second level information, transmit a first signal for controlling to display a first screen representing the health information of the user through a display of the first external electronic device.

### CROSS-REFERENCE TO RELATED APPLICATIONS

This application is a continuation of International Application No. PCT/KR2024/007806 designating the United States, filed on Jun. 7, 2024, in the Korean Intellectual Property Receiving Office and claiming priority to Korean Patent Application Nos. 10-2023-0108661, filed on Aug. 19, 2023, and 10-2023-0125963, filed on Sep. 20, 2023, in the Korean Intellectual Property Office, the disclosures of each of which are incorporated by reference herein in their entireties.

### BACKGROUND

### Field

The disclosure relates to an electronic device and a method for displaying information on a user through an external electronic device.

### Description of Related Art

With the development of technology, various electronic devices may operate in conjunction with each other. The electronic device may establish a connection with another electronic device. The electronic device may display information stored in the electronic device through a display of the other electronic device based on establishing the connection with the other electronic device.

The above-described information may be provided as a related art for the purpose of helping to understand the present disclosure. No claim or determination is raised as to whether any of the above-described information may be applied as a prior art related to the present disclosure.

### SUMMARY

According to an example embodiment, an electronic device may comprise: at least one sensor configured to detect health information of a user, a communication circuit configured to perform communication with an external device, memory including one or more storage mediums storing instructions and at least one processor comprising processing circuitry. The instructions, when being executed by the at least one processor individually or collectively, cause the electronic device to identify first level information of the health information of the user obtained based on the at least one sensor of the electronic device; identify second level information related to the first external electronic device, based on establishing a connection with a first external electronic device; transmit a first signal for controlling to display a first screen representing the health information of the user through a display of the first external electronic device, based on the first level information and the second level information.

According to an example embodiment, a method for displaying a screen representing health information through an external device may comprise: identifying first level information of health information of a user obtained based on at least one sensor of an electronic device; based on establishing a connection with a first external electronic device, identifying second level information related to the first external electronic device; and based on the first level information and the second level information, transmitting a first signal for controlling to display a first screen for representing the health information of the user through a display of the first external electronic device.

According to an example embodiment, an electronic device may comprise a communication circuit configured to perform communication with an external device, memory including one or more storage mediums storing instructions, and at least one processor comprising processing circuitry. The instructions, when being executed by the at least one processor individually or collectively, cause the electronic device to set first level information of health information of a user obtained based on at least one sensor of the first external electronic device and second level information related to a second external electronic device, based on establishing a connection with a first external electronic device; and transmit the first level information and the second level information to the first external electronic device.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment, according to various embodiments;

FIG. 2 is a diagram illustrating an example of an environment including a first electronic device, a second electronic device, and an external electronic device, according to various embodiments;

FIG. 3 is a block diagram illustrating an example configuration of a first electronic device and a second electronic device, according to various embodiments;

FIG. 4 is a partial cross-sectional view of a first electronic device, according to various embodiments;

FIG. 5 is a flowchart illustrating an example operation of a first electronic device, according to various embodiments;

FIG. 6 is a diagram illustrating an example of a first display level, according to various embodiments;

FIG. 7 is a diagram illustrating an example operation of a first electronic device, a second electronic device, and a first external electronic device, according to various embodiments;

FIG. 8 is a signal flow diagram illustrating an example operation of a first electronic device, a second electronic device, a first external electronic device, and a second external electronic device, according to various embodiments;

FIG. 9A is a signal flow diagram illustrating example operations between a first electronic device and a second electronic device, according to various embodiments;

FIG. 9B is a diagram illustrating an example of a user interface for setting a first display level, according to various embodiments;

FIG. 10 is a signal flow diagram illustrating example operations between a first electronic device and a second electronic device, according to various embodiments;

FIG. 11 is a flowchart illustrating an example operation of a first electronic device, according to various embodiments;

FIG. 12 is a flowchart illustrating an example operation of a first electronic device, according to various embodiments;

FIG. 13 is a diagram illustrating an example operation related to a designated model included in a second electronic device, according to various embodiments;

FIG. 14A is a diagram illustrating an example of a screen displayed at a first external electronic device in case that a designated model is not used, according to various embodiments;

FIG. 14B is a diagram illustrating an example of a screen displayed at a first external electronic device in case that a designated model is used, according to various embodiments;

FIG. 15A is a diagram illustrating an example operation of a first electronic device, a second electronic device, and an external electronic device, according to various embodiments;

FIG. 15B is a diagram illustrating an example operation of a first electronic device, a second electronic device, and an external electronic device, according to various embodiments; and

FIG. 16 is a diagram illustrating an example operation of a first electronic device, a second electronic device, and an external electronic device, according to various embodiments.

### DETAILED DESCRIPTION

Hereinafter, various example embodiments of the present disclosure will be described in greater detail with reference to drawings. However, the present disclosure may be implemented in various different forms and is not limited to the embodiments described herein. In relation to the description of the drawings, identical or similar reference numerals may be used for identical or similar components. In addition, in the drawings and related descriptions, descriptions of well-known features and configurations may be omitted for clarity and brevity.

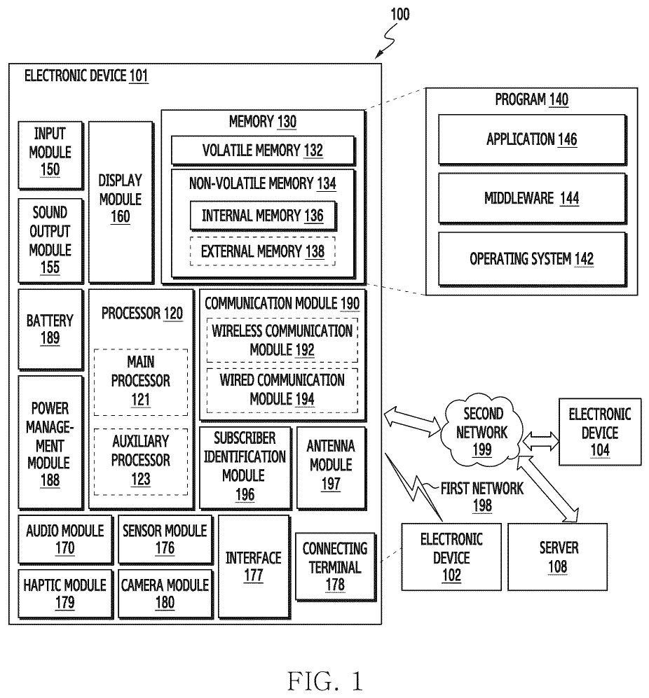

FIG. 1 is a block diagram illustrating an example electronic device in a network environment, according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), decp Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mm Wave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101.

The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

According to an embodiment, a first electronic device may identify (or detect) a user's physical activity, in a state worn on the user. For example, the first electronic device may identify (or obtain) information on the user (e.g., health information of the user) using a sensor, in a state worn on a part of the user's body (e.g., a finger). For example, the first electronic device may operate, in a state connected to a second electronic device (e.g., the electronic device 101 of FIG. 1). The first electronic device may display information on the user through an external electronic device, in a state connected to the second electronic device. For the user's privacy, the first electronic device may display at least a part of information on the user, or may change the information on the user and display the information through the external electronic device. According to an embodiment, the first electronic device may display information on the user on a display of the external electronic device through the second electronic device connected to the first electronic device. Operations of the first electronic device and/or the second electronic device according to the above-described embodiment will be described later. For convenience of description, a structure and an operation of the first electronic device formed in ring-shape will be described in the following specification. However, it is not limited thereto. For example, the first electronic device may be implemented in various forms capable of being worn by a user, such as a smart watch, a smart band, a smart ring, a wireless earphone, or a smart glass.

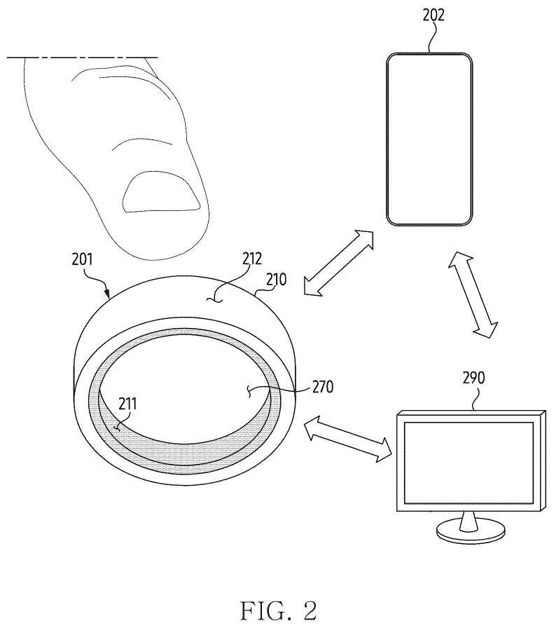

FIG. 2 is a diagram illustrating an example of an environment including a first electronic device, a second electronic device, and an external electronic device, according to various embodiments.

Referring to FIG. 2, a first electronic device 201 may include a housing 210 including a first surface 211 facing a part (e.g., a finger) of a user's body and a second surface 212 opposite to the first surface 211. For example, the first electronic device 201 may include a ring-shaped housing 210. For example, the first electronic device 201 may be configured in a ring shape.

According to an embodiment, the first electronic device 201 may be referred to as a wearable device capable of being worn by a user. The first electronic device 201 may be worn on a part (e.g., a finger) of the user's body. For example, the first electronic device 201 may be worn on a part of the user's body. For example, the first electronic device 201 may be fastened to a part of the user's body. For example, the first electronic device 201 may be detachable with respect to a part of the user's body. For example, the first electronic device 201 may have a shape corresponding to a part of the user's body, in order to be worn on the part of the user's body.

For example, the first electronic device 201 may be in contact with a part of the user's body by being worn by the user. For example, the first electronic device 201 may be configured to obtain information on the user through a part of the user's body by being worn on the user. For example, the information on the user may include health information of the user. However, the disclosure is not limited thereto. For example, the first electronic device 201 may provide information on the user through the first electronic device 201 and/or the second electronic device 202 connected to the first electronic device 201, or an external electronic device 290. However, the disclosure is not limited thereto.

According to an embodiment, at least a part of the first surface 211 may be in contact with a part of the user's body, when the first electronic device 201 is worn on the user. For example, the first surface 211 may surround a part of the user's body in which the first electronic device 201 is worn. For example, when the first electronic device 201 is worn on the user, the first surface 211 may be configured such that the first electronic device 201 is fastened to a part of the body by pressurizing a part of the user's body. For example, the first surface 211 may be deformable by a part of the user's body. For example, the first electronic device 201 may provide information on the user through the first surface 211, based on haptic technology.

For example, the second surface 212 may form the exterior of the first electronic device 201 together with the first surface 211. For example, the second surface 212 may form the ring-shaped housing 210 together with the first surface 211. For example, the second surface 212 may be a surface spaced apart from a part of the user's body when the first electronic device 201 is worn on the user. For example, the first surface 211 may be referred to as an inner circumference surface of the housing 210. The second surface 212 opposite to the first surface 211 may be referred to as an outer circumference surface of the housing 210.

For example, the second surface 212 may be exposed to the outside in a state in which the first electronic device 201 is worn by the user. The second surface 212 may include at least one of titanium, stainless steel, and ceramic. The second surface 212 may include a material for protecting against external impact and/or scratches. According to an embodiment, the second surface 212 may be coated with an additional material to protect the color of the first electronic device 201 and/or the exterior of the first electronic device 201.

For example, the first surface 211 may include the same and/or similar material as the second surface 212. According to an embodiment, at least a part of the first surface 211 may include at least one of a molding material, transparent plastic, and/or glass for obtaining data. According to an embodiment, at least a part of the first surface 211 may include a metal for identifying a bio-signal.

According to an embodiment, the first electronic device 201 may further include a hole 270 formed by the first surface 211 to pass a part of the user's body when the first electronic device 201 is worn by the user. For example, when the first electronic device 201 is worn by the user, the hole 270 may be penetrated by a part of the user's body. The first electronic device 201 may be configured to be fastened to a part of the user's body when the user wears the first electronic device 201, by including the hole 270 configured to pass through a part of the user's body.

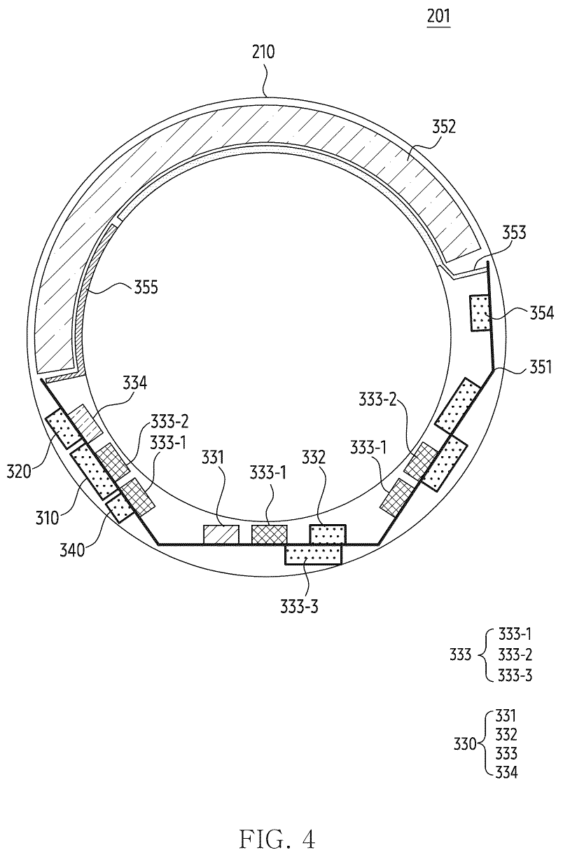

According to an embodiment, the first electronic device 201 may further include one or more components between the first surface 211 and the second surface 212. For example, the first electronic device 201 may include a communication circuit, one or more sensors, and/or a processor between the first surface 211 and the second surface 212. The arrangement of one or more components will be described later in FIG. 4.

According to an embodiment, the first electronic device 201 may establish a connection with the second electronic device 202 or the external electronic device 290, using various radio access technologies (RATs) (e.g., Bluetooth communication (or Bluetooth low energy (BLE) communication), wireless LAN, near field communication (NFC), ultra-wide band (UWB) communication)).

According to an embodiment, the first electronic device 201 may be connected to the second electronic device 202. For example, the user of the second electronic device 202 may be the same as the user of the first electronic device 201. As an example, the second electronic device 202 may be a device that is connected with the first electronic device 201 within a relatively short distance and used by the same user.

For example, the first electronic device 201 may control the second electronic device 202 or may be controlled by the second electronic device 202. Setting information of the first electronic device 201 may be changed by the second electronic device 202. The user may change the setting information of the first electronic device 201 using the second electronic device 202.

According to an embodiment, the first electronic device 201 may be connected with the external electronic device 290. For example, the external electronic device 290 may be used to display information (e.g., information on the user) of the first electronic device 201. The external electronic device 290 may include a display for displaying information of the first electronic device 201. The first electronic device 201 may transmit a signal for controlling to display information on the user using the display of the external electronic device 290 to the external electronic device 290, based on establishing a connection with the external electronic device 290. The external electronic device 290 may display information on the user through the display, based on the received signal.

According to an embodiment, the first electronic device 201 may transmit a signal for controlling to display information on the user using the display of the external electronic device 290 to the second electronic device 202. The second electronic device 202 may generate a screen for representing information on the user. The second electronic device 202 may transmit information on the generated screen to the external electronic device 290. Based on the received information on the screen, the external electronic device 290 may display a screen for representing information on the user.

Embodiments described in greater detail below will be described through the first electronic device 201 and the second electronic device 202 described above. However, the disclosure is not limited thereto.

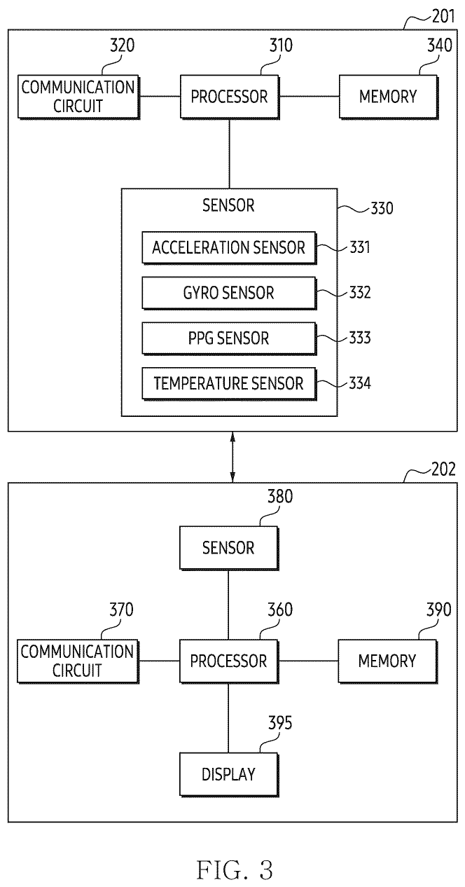

FIG. 3 is a block diagram illustrating an example configuration of a first electronic device and a second electronic device, according to various embodiments.

Referring to FIG. 3, a first electronic device 201 and a second electronic device 202 may operate in a connected state. For example, the first electronic device 201 may be used to obtain (or identify) information on a user. The second electronic device 202 may be used to control the first electronic device 201.

According to an embodiment, the first electronic device 201 may include a processor (e.g., including processing circuitry) 310, a communication circuit 320, a sensor 330, and/or a memory 340. According to an embodiment, the first electronic device 201 may include at least one of the processor 310, the communication circuit 320, the sensor 330, and/or the memory 340. For example, at least a part of the processor 310, the communication circuit 320, the sensor 330, and/or the memory 340 may be omitted according to an embodiment. For example, the first electronic device 201 may include at least a part of the components of the electronic device 101 of FIG. 1.

According to an embodiment, the processor 310 may correspond to the processor 120 of FIG. 1. The processor 310 may include various processing circuitry and be operably coupled or connected with the communication circuit 320, the sensor 330, and the memory 340. The processor 310 being operably coupled or connected with the communication circuit 320, the sensor 330, and the memory 340 may refer, for example, to the processor 310 being configured to control the communication circuit 320, the sensor 330, and the memory 340. For example, the communication circuit 320, the sensor 330, and the memory 340 may be controlled by the processor 310.

Although illustrated based on different blocks, embodiments are not limited thereto, and a part of the hardware of FIG. 3 (e.g., at least a part of the processor 310, the communication circuit 320, and the memory 340) may be included in a single integrated circuit, such as a system on a chip (SoC).

According to an embodiment, the processor 310 may be configured with at least one processor. For example, the processor 310 may comprise a main processor performing high-performance processing and an auxiliary processor performing low-power processing. At least a part of the sensors 330 may be connected to the auxiliary processor. At least a part of the sensors connected to the auxiliary processor may obtain data on the user for 24 hours. According to an embodiment, one of the main processor and the auxiliary processor may be activated according to a state and/or operation of the first electronic device 201. For example, in a state in which a battery of the first electronic device 201 is insufficient, the auxiliary processor may be activated. For example, in a state in which accurate data on the user is required, the main processor may be activated.

According to an embodiment, the processor 310 may include a hardware component for processing data based on one or more instructions. For example, hardware components for processing data may include an arithmetic and logic unit (ALU), a field programmable gate array (FPGA), and/or a central processing unit (CPU).

For example, the processor 310 may include an application processor, a supplementary processor (e.g., a sensor hub, a microcontroller unit (MCU)), a central processor unit (CPU), a neural processing unit (NPU), a graphic processing unit (GPU), and/or a processor for IoT (e.g., a processor integrated with a communication module). The processor 310 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

According to an embodiment, the processor 310 may determine an operation timing of the sensor 330. The processor 310 may control an operation of the sensor 330. The processor 310 may process information obtained from the sensor 330.

According to an embodiment, the first electronic device 201 may include a communication circuit 320. The communication circuit 320 may correspond to at least a part of the communication module 190 of FIG. 1. For example, the communication circuit 320 may be used for various radio access technologies (RATs). For example, the communication circuit 320 may be used to perform Bluetooth communication, wireless local area network (WLAN) communication, Zigbee communication, near field communication (NFC), ultra-wide band (UWB) communication, or ANT+ communication. For example, the communication circuit 320 may be used to perform cellular communication. For example, the processor 310 may establish a connection with another electronic device (e.g., the second electronic device 202 or the external electronic device 290 of FIG. 2) through the communication circuit 320. For example, the processor 310 may identify (or measure) a location of the first electronic device 201, based on a received or transmitted wireless signal (e.g., a global positioning system (GPS) signal) using the communication circuit 320. According to an embodiment, the communication circuit 320 may be configured to be integrated with the processor 310.

According to an embodiment, the first electronic device 201 may include the sensor 330. The sensor 330 may be used to obtain various information. For example, the sensor 330 may be used to obtain information on the user. The information on the user may include data on the user's body.

As an example, the sensor 330 may be used to obtain the user's body temperature data (or body temperature information), heart rate data (or heart rate information), and/or motion data (or motion information). For example, the sensor 330 may be configured with at least one sensor. The sensor 330 may include at least one sensor. For example, the sensor 330 may correspond to the sensor module 176 of FIG. 1.

For example, the sensor 330 may include an acceleration sensor 331. The acceleration sensor 331 may be used to identify a change in the acceleration of the first electronic device 201. As an example, the acceleration sensor 331 may identify (or measure, detect) acceleration of the first electronic device 201 in three directions of the x-axis, y-axis, and z-axis. As an example, the acceleration sensor 331 may be set to identify acceleration of about four times greater than or equal to the acceleration of gravity at an arbitrary axis. As an example, the acceleration sensor 331 may have resolution of a designated size (e.g., 16 bits).

For example, the sensor 330 may include a gyro sensor 332. The gyro sensor 332 may identify (or measure, detect) angular velocity of the first electronic device 201 in three directions of the x-axis, y-axis, and z-axis. According to an embodiment, the first electronic device 201 may include an inertial sensor including the acceleration sensor 331 and the gyro sensor 332.

For example, the first electronic device 201 may identify at least one of data related to tapping, data related to a user's gesture, data related to the amount of impact, data related to the orientation of the first electronic device 201, and/or activity information (e.g., sitting, moving, sports activities) of the user, using at least one of the acceleration sensor 331 and/or the gyro sensor 332.

For example, the sensor 330 may include a photoplethysmography (PPG) sensor 333. The PPG sensor 333 may be used to measure a pulse (or a change in the amount of blood in a blood vessel) by identifying a change in the amount of photosensitization of light according to a change in blood vessel volume. The PPG sensor 333 may include one or more photodiodes (PDs) and one or more light emitting diodes (LEDs). For example, the PPG sensor 333 may be used to identify a change in blood flow amount in a blood vessel during a heartbeat. The PPG sensor 333 may identify a change in blood flow amount in a blood vessel during a heartbeat, in a state in which a light sensor is in contact with a skin above peripheral blood vessels. The processor 310 may identify blood flow amount based on a PPG signal and waveform, and identify the amount of change in the blood flow amount.

For example, the PPG sensor 333 may include a transmissive PPG sensor and/or a reflective PPG sensor.

For example, the PPG sensor 333 may output light toward the user's skin, through one of LED (e.g., green, red, or infrared (IR)), laser, and vertical cavity surface emitting laser (VCSEL). The PPG sensor 333 may identify light reflected and/or penetrated from the user's skin through at least one of PD and/or complementary metal oxide semiconductor (CMOS) camera. The PPG sensor 333 may store a value identified through an analog to digital converter (ADC) in the memory 340 (or buffer), based on the reflected and/or penetrated light.

As an example, the transmissive PPG sensor may identify light penetrated through a blood vessel, through PD disposed on an opposite side of the LED. The transmissive PPG sensor may identify the blood flow amount of the user, based on intensity of light penetrated through the blood vessel. For example, the reflective PPG sensor may output light toward the user's skin through the LED. The reflective PPG sensor may identify at least partially received light by reflecting from the blood vessel, through PD disposed on substantially the same plane as the LED. The reflective PPG sensor may identify the blood flow amount of the user, based on intensity of light reflected by the blood vessel. For example, a multi-light source may be used as the LED. For example, the LED may use green light, which is a complementary color to blood.

For example, the sensor 330 may include a temperature sensor 334. The temperature sensor 334 may be used to identify (or measure) a skin temperature at a part of the user's body (e.g., wrist or forehead). For example, the temperature sensor 334 may include a contact type temperature sensor and a non-contact type temperature sensor.

Although not illustrated, the sensor 330 may further include various sensors for obtaining (or identifying, measuring, and detecting) various data related to the user and/or the first electronic device 201.

According to an embodiment, the first electronic device 201 may include the memory 340. The memory 340 may be used to store information or data. For example, the memory 340 may be used to store data obtained from the user. For example, the memory 340 may correspond to the memory 130 of FIG. 1. For example, the memory 340 may be a volatile memory unit or units. For example, the memory 340 may be a non-volatile memory unit or units. For another example, the memory 340 may be another form of computer-readable medium, such as a magnetic or optical disk. For example, the memory 340 may store data obtained based on an operation (e.g., an algorithm performing operation) performed by the processor 310. For example, the memory 340 may store data (e.g., information on the user) obtained by the sensor 330. According to an embodiment, the memory 340 may be configured in a form integrated with the processor 310.

Although not illustrated in FIG. 3, the first electronic device 201 may further include various components. For example, the first electronic device 201 may further include at least one of an antenna, a power management integrated circuit (PMIC), a battery, and a flexible printed circuit board (FPCB). According to an embodiment, the first electronic device 201 may further include a biometric sensor including the PPG sensor 333. The configuration and/or operation of the biometric sensor will be described in greater detail below.

According to an embodiment, the second electronic device 202 may include a processor (e.g., including processing circuitry) 360, a communication circuit 370, a sensor 380, a memory 390, and/or a display 395. According to an embodiment, the second electronic device 202 may include at least one of the processor 360, the communication circuit 370, the sensor 380, the memory 390, and/or the display 395. For example, at least a part of the processor 360, the communication circuit 370, the sensor 380, the memory 390, and/or the display 395 may be omitted according to an embodiment. For example, the second electronic device 202 may correspond to the electronic device 101 of FIG. 1. For example, the second electronic device 202 may include at least a part of the components of the electronic device 101 of FIG. 1.

For example, the processor 360 may correspond to the processor 310 of the first electronic device 201. For example, the communication circuit 370 may correspond to the communication circuit 320 of the first electronic device 201. For example, the memory 390 may correspond to the memory 340 of the first electronic device 201.

According to an embodiment, the sensor 380 may be used to obtain various information. According to an embodiment, the sensor 380 may obtain information distinguished from information obtained by the sensor 330 of the first electronic device 201.

For example, the sensor 380 may include a biometric sensor (e.g., a biometric optical sensor). For example, the biometric sensor may include a PPG sensor (e.g., the PPG sensor 333) and/or an electrocardiogram (ECG) sensor.

According to an embodiment, the biometric sensor (e.g., the PPG sensor) may include a light source circuit, a light source detection circuit, and an integrated circuit (IC). The light source circuit may include at least one light emitting diode (LED) having a plurality of wavelength bands. A signal having a wavelength band related to green may penetrate thinly into the skin and be strong against noise. Therefore, the signal having the wavelength band related to green may be used to obtain information on heart rate. A signal having an infrared radiation (IR) wavelength band may be used to obtain biometric information including heart rate and/or saturation pulse oxygen (SpO2) together with a signal having a wavelength band related to red.

The signal having a wavelength band related to red, the signal having a wavelength band related to green, and the signal having an IR wavelength band may be used to identify the skin tone. When a signal having a wavelength band related to blue is added, the signals may be used to identify a trend of blood sugar. As described above, as signals of various wavelength bands are added, more biometric information may be obtained.

For example, the light source circuit may be configured to emit a signal of one or more wavelength bands. The light source circuit may include at least one emitter for each wavelength band.

The light source detection circuit may include at least one photodiode (PD). A first PD among the at least one PD may be disposed to be spaced apart from the light source circuit by a first distance. A second PD may be disposed to be spaced apart from the light source circuit by a second distance.

A sensor IC may include a sensor driver controller that directly controls a sensor and an analog-to-digital converter (ADC). The sensor driver controller may include a light source circuit controller and a light source detection circuit controller. The light source circuit controller may be configured to control (e.g., driving) the light source circuit. The light source detection circuit controller may be configured to control (e.g., driving) the light source detection circuit.

The sensor driver controller may be configured to perform an operation of analog front end (AFE). The sensor driver controller may include the AFE. For example, the AFE may include an amplifier for amplifying values (or data) obtained from LED drivers and the light source detection circuit, the ADC for converting analog values obtained from the light source detection circuits into digital values, and a controller for controlling the LED drivers and the ADC. According to an embodiment, the sensor driver controller may not include the AFE. The sensor driver controller may be configured to perform an operation of the AFE through a circuit including the amplifier, the ADC, and a switch.

For example, data on light (e.g., photo) identified through the light source detection circuit may be transmitted to the processor 360 through at least one filter and the ADC. The processor 360 may obtain information (or biometric information) on the user, based on the data on light (e.g., photo). The processor 360 may provide information on the user to the user. The processor 360 may store information on the user through an application. The processor 360 may transfer (or transmit) information on the user to a 3rd party.

According to an embodiment, the biometric sensor (e.g., the ECG sensor) may include an electrode for identifying at least one of the electrocardiogram (ECG), galvanic skin response (GSR), bioelectrical impedance analysis (BIA), and/or electroencephalography (EEG). For example, the processor 360 may identify at least one of the ECG, the GSR, the BIA, and/or the EEG, based on an electrical signal obtained through the electrode.

According to an embodiment, the sensor 380 may include at least one of an acceleration sensor, a gyro sensor, a proximity sensor, a body temperature sensor, and/or an iris sensor. According to an embodiment, the sensor 380 may include at least one of a temperature sensor, a humidity sensor, an illuminance sensor, and/or a time of flight (TOF) sensor, in order to obtain information on an external environment. According to an embodiment, the sensor 380 may include at least one of a gas sensor and/or a fine dust sensor.

According to an embodiment, the display 395 may output visualized information to the user. For example, the display 395 may be controlled by the processor 360 including a circuit such as a graphic processing unit (GPU) to output visualized information to the user. For example, the display 395 may correspond to the display module 160 of FIG. 1.

For example, the display 395 may be used to display a screen for representing information on the user. For example, the display 395 may be used to display a user interface for setting a display level of information on the user obtained from the first electronic device 201.

FIG. 4 is a partial cross-sectional view of a first electronic device, according to various embodiments.

Referring to FIG. 4, a first electronic device 201 may be formed in a ring shape. For example, a housing 210 of the first electronic device 201 may be formed in the ring shape capable of being worn on a user's finger. FIG. 2 or FIG. 4 illustrates the first electronic device 201 having a ring shape with a smooth surface as an example, but the disclosure is not limited thereto. For example, the first electronic device 201 may be implemented as a housing including a plurality of planes. For example, the first electronic device 201 having a ring shape with a non-smooth surface may also be understood as an embodiment of the present disclosure.

According to an embodiment, the ring-shaped housing 210 may include a first surface (e.g., the first surface 211 of FIG. 2) in contact with a user's body in a state worn by the user, a second surface (e.g., the second surface 212 of FIG. 2) exposed to the outside, and a side surface between the first surface 211 and the second surface 212. For example, a space for including (or placing) at least one component (e.g., the processor 310, the communication circuit 320, the sensor 330, and the memory 340) may be included between the first surface 211 and the second surface 212.

According to an embodiment, a PCB 351 may be disposed between the first surface 211 and the second surface 212 of the first electronic device 201. For example, a processor 310, a communication circuit 320, an acceleration sensor 331, a gyro sensor 332, a PPG sensor 333, a temperature sensor 334, a memory 340, and/or a PMIC 354 may be disposed on the PCB 351. For example, the PCB 351 may include a rigid region and a flexible region. For example, the rigid region may be referred to as a rigid flexible printed circuit board (RFPCB). For example, the flexible region may be referred to as a flexible printed circuit board (FPCB).

For example, the PPG sensor 333 may include one or more light emitting circuit 333-1, one or more light receiving circuit 333-2, and a control circuit 333-3. As an example, one or more light emitting circuit 333-1 and one or more light receiving circuit 333-2 may be disposed toward the first surface 211. As an example, the control circuit 333-3 may be disposed toward the second surface 212.

For example, the PMIC 354 may be used to manage power of the first electronic device 201. The PMIC 354 may be used in the first electronic device 201 to provide (or distribute) power to components requiring power. The PMIC 354 may support a wired charging method (e.g., terminal, pogo pin) or a wireless charging method (e.g., a wireless power consortium (WPC), NFC) for charging the first electronic device 201 through a charging interface 353.

According to an embodiment, a battery 352 may be disposed between the first surface 211 and the second surface 212 of the first electronic device 201. The battery 352 may be configured with at least one battery (or battery pack). For example, the battery 352 may be configured such that at least one battery is connected in series and/or parallel. For example, the battery 352 may be configured with a flexible battery pack. For example, the battery 352 may be charged and/or discharged with a secondary battery. For example, a material configuring the battery 352 may be variously configured. For example, the material configuring the battery 352 may include at least one of lithium ion and mercury.

According to an embodiment, an antenna 355 may be disposed between the first surface 211 and the second surface 212 of the first electronic device 201. For example, the antenna 355 may configured with a single antenna and/or a plurality of segmented antennas. According to an embodiment, the antenna 355 may be configured as a part of the housing 210 of the first electronic device 201. For example, the antenna 355 may be electrically connected with the communication circuit 320 through the PCB 351.

Although not illustrated, the first electronic device 201 may further include various components in addition to the illustrated components. For example, the first electronic device 201 may include a display. The display may be disposed on an outer surface of the housing 210.

According to an embodiment, the first electronic device 201 may display information on the user through the display of a first external electronic device (e.g., the external electronic device 290 of FIG. 2). The first electronic device 201 may identify the access of a second external electronic device while a first screen for representing information on the user is displayed through the display of the first external electronic device. Based on identifying the access of the second external electronic device, the first electronic device 201 may change the first screen for representing information on the user through the display of the first external electronic device to the second screen. In the following description, an operation of the first electronic device 201 according to the above-described embodiment may be described.

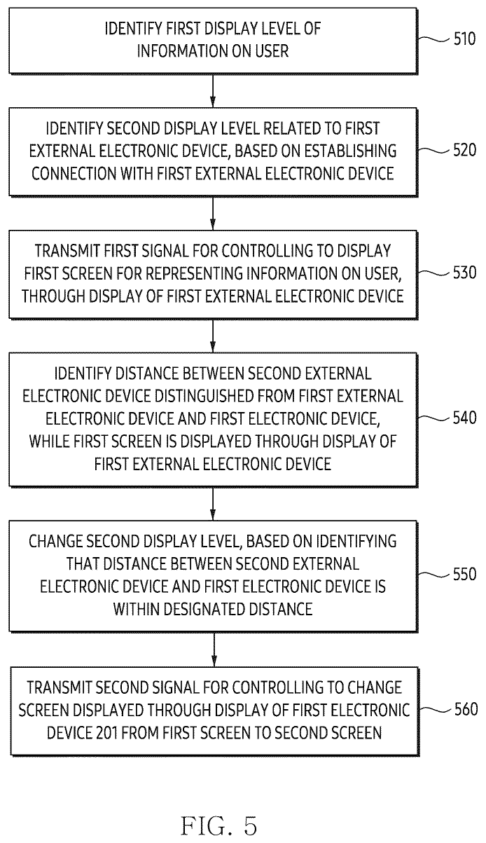

FIG. 5 is a flowchart illustrating an example operation of a first electronic device, according to various embodiments. In the following embodiment, each operation may be performed sequentially, but is not necessarily performed sequentially. For example, the sequence of each operation may be changed, and at least two operations may be performed in parallel.

Referring to FIG. 5, in operation 510, a processor 310 may identify a first display level of information on a user. For example, the processor 310 may identify the first display level of information on the user obtained through a sensor 330. According to an embodiment, the processor 310 may obtain (or identify) information on the user through the sensor 330. For example, the information on the user may include at least one of the user's body information, activity information, real-time exercise information, sleep information, women's health information, and dietary intake information.

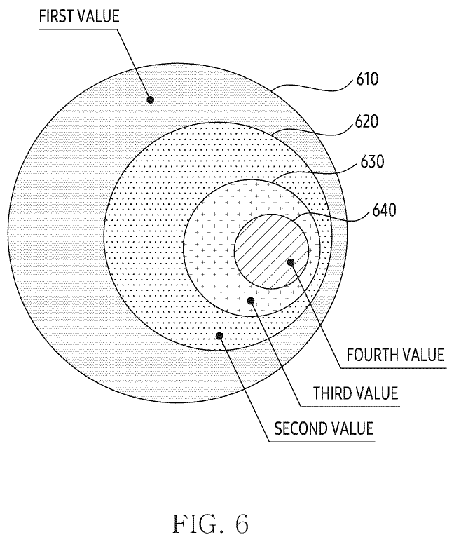

For example, the information on the user may be identified as one of a plurality of types. As an example, the plurality of types may include a fitness type, a wellness type, and a medical type. The information on the user may be identified as one of the fitness type, the wellness type, and the medical type. A detailed example of information on the user and a display level according to the information on the user will be described later in FIG. 6.

For example, the processor 310 may store information on the user in the memory 340 in conjunction with the first display level. The first display level may identify display levels for each of the user's body information, activity information, real-time exercise information, sleep information, women's health information, and dietary intake information. The processor 410 may store display levels for each of the user's body information, activity information, real-time exercise information, sleep information, women's health information, and dictary intake information in the memory 340, in conjunction with identified display levels.

According to an embodiment, the first display level of information on the user and a second display level of the first external electronic device may be set. The first display level and the second display level may be distinguished from each other.

For example, the first display level may represent sensitivity of information on the user. For example, the first display level may represent an exposure reference of information on the user. The lower the first display level, the higher the exposure reference may be set. The higher the first display level, the lower the exposure reference may be set. For example, the first display level may be set to one of a first value to a fourth value. Information on the user whose the first display level is set to the first value may be set to be limitedly displayed so that only the user can see it. Information on the user whose the first display level is set to the fourth value may be set to be exposed to people other than the user. As an example, a first display level of information on the user's weight may be set to the first value. A first display level of information on the user's age may be set to the fourth value.

For example, the information on the user may include sensitive personal information of the user. Accordingly, the first display level may be set according to information on the user, in order to protect the user's privacy.

For example, the second display level may represent a reference of information displayable at the first external electronic device. As an example, the first external electronic device may be set to display information on the user having a display level higher than the second display level. As an example, the first external electronic device may be set to not display information on the user having a display level lower than the second display level, or limitedly display. As an example, when the first external electronic device is a commonly used device, the display level of the first external electronic device may be set to be high. As an example, when the first external electronic device is a personally used device, the display level of the first external electronic device may be set to be low.

The above-described first display level and second display level may be referred to as a privacy level, in terms of being set to protect the privacy of the user.

According to an embodiment, the processor 310 may identify the first display level of information on the user. The first display level of information on the user may be a reference for determining (or identifying) whether to limit the display of information on the user. For example, when information on the user is displayed on a display (e.g., a display of the first external electronic device), the processor 310 may identify (or determine) whether information on the user will be displayed, based on the first display level and the second display level.

In operation 520, the processor 310 may identify the second display level related to the first external electronic device, based on establishing a connection with the first external electronic device. For example, the processor 310 may identify the second display level related to the first external electronic device, based on establishing a connection with the first external electronic device including the display to display information on the user.

According to an embodiment, the processor 310 may establish a connection with the first external electronic device. For example, the first external electronic device may include a display. The first external electronic device may be used to display information on the user received from the first electronic device 201.

According to an embodiment, the processor 410 may identify (or check) the first display level stored in a memory 415, in order to compare the first display level with the second display level.

According to an embodiment, the processor 310 may identify the second display level related to the first external electronic device. The processor 310 may identify the second display level related to the first external electronic device in order to compare the first display level with the second display level. For example, based on a connection with the first external electronic device, the processor 310 may obtain (or receive) at least one of information on an account of the first external electronic device, information on an identifier of the first external electronic device, information on a location of the first external electronic device, and/or information on a connection history of the first external electronic device, from the first external electronic device. The processor 310 may identify the second display level, based on the at least one of information on an account of the first external electronic device, information on an identifier of the first external electronic device, information on a location of the first external electronic device, and/or information on a connection history of the first external electronic device. For example, the second display level may not be a fixed value. The second display level may be changed based on a change in at least a part of information for identifying the second display level.

In operation 530, the processor 310 may transmit a first signal for controlling to display a first screen for representing information on the user, through the display of the first external electronic device. For example, based on the first display level and the second display level, the processor 310 may transmit the first signal for controlling to display the first screen for representing information on the user, through the display of the first external electronic device.

According to an embodiment, the processor 310 may request information on the first screen from the second electronic device 202. The second electronic device 202 (e.g., the processor 360 of the second electronic device 202) may generate (or obtain) the first screen using a designated model, based on the received request. For example, the first screen may be generated (or obtained) through the designated model.

For example, the second electronic device 202 may include the designated model for generating (or obtaining) a screen for representing information on the user. The designated model may be configured based on a plurality of parameters. For example, the designated model may be a model trained according to a supervised learning method based on an artificial intelligence algorithm or a model trained according to an unsupervised learning method. The trained model may be configured to include a plurality of network nodes having weights, and the plurality of network nodes may transmit and receive data according to a convolution connection relationship while being located at different depths (or layers). For example, a deep neural network (DNN), a recurrent neural network (RNN), a bidirectional recurrent deep neural network (BRDNN), a generative adversarial network (GAN) may be used as a learning model, but are not limited thereto. For example, the designated model may be configured based on a generative artificial intelligence (AI) model.

For example, the first display level and the second display level may be set as an input value of the designated model. The screen (e.g., the first screen) for representing information on the user may be set as an output value of the designated model. The second electronic device 202 may set the first display level and the second display level as an input value of the designated model. Based on the output value of the designated model, the second electronic device 202 may obtain the screen (e.g., the first screen) for representing information on the user. According to an embodiment, various information in addition to the first display level and the second display level may be set as the input value of the designated model. For example, at least one of the user's profile information, information on output setting (e.g., resolution, screen size, scan rate) of the first external electronic device, information on the location of the first external electronic device, information on a space where the first external electronic device is located, information on the account of the first external electronic device, information on the identifier of the first external electronic device, and/or information on the connection history of the first external electronic device may be set as the input value of the designated model.

In the above-described example, an example in which the designated model is included in the second electronic device 202 is described, but is not limited thereto. According to an embodiment, the designated model may be included in another device (e.g., server) distinguished from the second electronic device 202. The second electronic device 202 may transmit information for setting as the input value of the designated model to the other device (e.g., server). The other device may set the information received from the second electronic device 202 as an input value of a designated model included in the other electronic device. The other device may identify information on the first screen based on the output value of the designated model. The other device may transmit information on the first screen to the second electronic device 202. The second electronic device 202 may receive information on the first screen from the other device (e.g., server).

According to an embodiment, the second electronic device 202 may transmit information on the first screen to the first electronic device 201. The first electronic device 201 may transmit a first signal for controlling to display the first screen to the first external electronic device, based on the information on the first screen.

According to an embodiment, the second electronic device 202 may display the generated first screen through the display of the first external electronic device. The second electronic device 202 may transmit a signal for controlling to display the first screen to the first external electronic device. The first external electronic device may display the first screen based on the signal received from the second electronic device 202.

According to an embodiment, the processor 310 may perform at least part or all of operations of the second electronic device 202 described above. According to an embodiment, the processor 310 may generate the first screen using the designated model, and transmit the first signal for controlling to display the first screen to the first external electronic device.

According to an embodiment, the first external electronic device may include the designated model. Based on receiving the first signal, the first external electronic device may generate (or obtain) the first screen using the designated model. The first external electronic device may also display the first screen generated by itself, through the display.

In operation 540, while the first screen is displayed through the display of the first external electronic device, the processor 310 may identify a distance between the second external electronic device distinguished from the first external electronic device and the first electronic device 201. For example, the processor 310 may identify the distance between the second external electronic device and the first electronic device 201, based on a signal transmitted (or emitted) from the second external electronic device. As an example, the processor 310 may identify the distance between the second external electronic device and the first electronic device 201, based on the strength (e.g., received signal strength indicator (RSSI)) of a signal transmitted from the second external electronic device.

In operation 550, the processor 310 may change the second display level, based on identifying that the distance between the second external electronic device and the first electronic device 201 is within a designated distance.

According to an embodiment, the processor 310 may change the second display level, based on identifying that the distance between the second external electronic device and the first electronic device 201 is within the designated distance. Based on the change in the second display level, the second screen may be generated in the second electronic device 202.

For example, the processor 310 may identify a third display level of the second external electronic device. The processor 310 may change the second display level so that the second display level corresponds to the third display level.

According to an embodiment, the processor 310 may identify that the distance between the second external electronic device and the first electronic device 201 is within the designated distance. For example, the processor 310 may identify that the second external electronic device is approaching the first electronic device 201. Based on identifying that the second external electronic device is approaching the first electronic device 201, the processor 310 may identify that the distance between the second external electronic device and the first electronic device 201 is within the designated distance.

For example, the designated distance may be set as a visibility distance with respect to a screen (or content within the screen) displayed through the display of the first external electronic device. The processor 310 may change the second display level so that the user of the second external electronic device does not see the first screen.

In operation 560, the processor 310 may transmit a second signal for controlling to change the screen displayed through the display of the first electronic device 201 from the first screen to the second screen. For example, based on changing the second display level, the processor 310 may transmit the second signal for controlling to change the screen displayed through the display of the first electronic device 201 from the first screen to the second screen.

According to an embodiment, the processor 310 may transmit the second signal to the first external electronic device. For example, the processor 310 may receive information on the second screen from the second electronic device 202. The processor 310 may transmit the second signal including information on the second screen to the first external electronic device. The first external electronic device may change (or switch) the screen displayed through the display from the first screen to the second screen, based on the second signal. According to an embodiment, when the designated model is included in the first electronic device 201, the processor 310 may generate the second screen based on the designated model. The processor 310 may transmit the second signal including information on the second screen to the first external electronic device.

According to an embodiment, the processor 310 may transmit the second signal to the second electronic device 202. Based on the second signal, the second electronic device 202 may generate the second screen using the designated model. The second electronic device 202 may transmit a signal for controlling to display the second screen to the first external electronic device. The first external electronic device may change (or switch) the screen displayed through the display from the first screen to the second screen, based on the received signal.

According to an embodiment, the processor 310 may transmit a third signal for controlling to display a third screen representing that the screen displayed through the display of the first external electronic device is being changed, while the screen displayed through the display of the first external electronic device is changed from the first screen to the second screen. The first external electronic device may change the screen displayed through the display from the first screen to the third screen. The first external electronic device may change the third screen to the second screen, based on the change in the second display level of the first external electronic device.

FIG. 6 is a diagram illustrating an example of a first display level, according to various embodiments.

Referring to FIG. 6, a processor 310 may identify a first display level of information on a user (e.g., health information of the user). For example, the processor 310 may transmit a signal for requesting display of a screen for setting a first display level to a second electronic device 202, based on a connection with the second electronic device 202. The second electronic device 202 may display a user interface for setting the first display level through a display 395 of the second electronic device 202, based on the received signal. Based on the user interface displayed through the display 395 of the second electronic device 202, the first display level of information on the user may be set. The processor 310 may identify the first display level of the user information set at the second electronic device 202.

According to an embodiment, the first display level may refer to an exposure level of information to be displayed on an external electronic device (e.g., a first external electronic device). However, the disclosure is not limited thereto. For example, the higher the first display level, the higher the exposure level of information to be displayed on the external electronic device (e.g., the first external electronic device).

For example, data on the user according to the first display level may be displayed based on the second display level of the first external electronic device. For example, data on the user of a first display level value higher than a second display level value may be displayed at the first external electronic device.

According to an embodiment, the first display level may be set to one of a first value to a fourth value. The first to fourth values are examples, and the first display level (or the second display level) may be variously set. For example, information on the user may be set to be displayed only at a personal device, based on the first display level set to the first value (e.g., 1). For example, information on the user may be set to be possible to be displayed at a device of family member, based on the first display level set to the second value (e.g., 2). Information on the user may be set to be possible to be displayed at a device of a friend (or an acquaintance), based on the first display level set to the third value (e.g., 3). Information on the user may be set to be possible to be displayed at a public device, based on the first display level set to the fourth value (e.g., 4).

Ranges 610, 620, 630, and 640 illustrated in FIG. 6 may indicate a range of information on the user (e.g., health information of the user) capable of being displayed according to the second display level.

For example, according to the second display level set to the fourth value, a range of information on the user capable of being displayed at the first external electronic device may be set to a fourth range 640. When the second display level related to the first external electronic device is set to the fourth value, information on the user according to the first display level set to a value greater than or equal to the fourth value may be displayed. Based on the second display level set to the fourth value, only information on the user whose the first display level is set to the fourth value may be displayed through the display of the first external electronic device. For example, based on the second display level set to the fourth value, information capable of being displayed (or exposed) at a public device may be displayed through the display of the first external electronic device.

For example, according to the second display level set to the third value, the range of information on the user capable of being displayed in the first external electronic device may be set to a third range 630. The third range 630 may include the fourth range 640. When the second display level related to the first external electronic device is set to the third value, information on the user according to the first display level set to a value greater than or equal to the third value may be displayed. Based on the second display level set to the third value, information on the user whose the first display level is set to one of the third value and the fourth value may be displayed through the display of the first external electronic device. For example, based on the second display level set to the third value, information capable of being displayed (or exposed) at a device of a friend (or acquaintance) and information capable of being displayed (or exposed) at a public device may be displayed through the display of the first external electronic device.

For example, according to the second display level set to the second value, the range of information on the user capable of being displayed at the first external electronic device may be set to a second range 620. The second range 620 may include the third range 630. When the second display level related to the first external electronic device is set to the second value, information on the user according to the first display level set to a value greater than or equal to the second value may be displayed. Based on the second display level set to the second value, information on the user whose the first display level is set to one of the second value, the third value, and the fourth value may be displayed through the display of the first external electronic device. For example, based on the second display level set to the second value, information capable of being displayed (or exposed) at a device of a family member, information capable of being displayed (or exposed) at a device of a friend (or acquaintance), and information capable of being displayed (or exposed) on a public device may be displayed through the display of the first external electronic device.

For example, according to the second display level set to the first value, the range of information on the user capable of being displayed at the first external electronic device may be set to a first range 610. The first range 610 may include the second range 620. When the second display level related to the first external electronic device is set to the first value, information on the user according to the first display level set to a value greater than or equal to the first value may be displayed. Based on the second display level set to the first value, information on the user whose the first display level is set to one of the first value, the second value, the third value, and the fourth value may be displayed through the display of the first external electronic device. For example, based on the second display level set to the first value, information (e.g., personal sensitive information or health information) capable of being displayed (or exposed) on a personal device, information capable of being displayed (or exposed) on a device of a family member, information capable of being displayed (or exposed) on a device of a friend (or acquaintance), and information capable of being displayed (or exposed) on a public device may be displayed through the display of the first external electronic device.

According to an embodiment, the information on the user may be identified as one of a plurality of types. For example, the processor 310 may identify the information on the user as one of the plurality of types. For example, the plurality of types may include a fitness type, a wellness type, and a medical type. However, the disclosure is not limited thereto.

For example, a display level may be set in each of the plurality of types. Display levels of each of the plurality of types may be set based on a user input. As an example, a user interface for setting display levels of each of the plurality of types may be displayed through the display 395 of the second electronic device 202. The second electronic device 202 may set display levels of each of the plurality of types, based on a user input identified while the user interface is being displayed.

For example, the display levels of each of the plurality of types may be set as shown in the following table.

TABLE 1

| Type | Display level |
|---|---|
| Fitness | 4 |
| Wellness | 2 |
| Medical | 1 |

Referring to Table 1, a first display level of information on a fitness type user may be set to a fourth value (e.g., 4). The information on the fitness type user may be set to be possible to be displayed at a public device. A first display level of information on a wellness type user may be set to a second value (e.g., 2). The information on the wellness type user may be set to be possible to be displayed at a device of a family member. A first display level of information on a medical-type user may be set to a first value (e.g., 1). The information on the medical-type user may be set to be possible to be displayed at a personal device.

According to an embodiment, a display level of data included in each of the plurality of types may be set. The display level of data included in each of the plurality of types may be predefined. According to an embodiment, the display level of data included in each of the plurality of types may be predefined according to country in which the first electronic device 201 is used, model of the first electronic device 201, and/or the law. However, the disclosure is not limited thereto. According to an embodiment, the display level of data included in each of the plurality of types may be changed based on a user input.

Table 2, Table 3 and Table 4 may be examples of data included in information on the fitness type user.

For example, a display level of data related to real-time exercise information among the information on the fitness type user may be set as shown in Table 2.

TABLE 2

| Data | Display level |
|---|---|
| Exercise time | 3 |
| Exercise intensity | 2 |
| Exercise type | 1 |
| Calories burned | 1 |
| Heart rate | 1 |
| Sweat loss | 1 |
| Oxygen saturation | 1 |
| Vo2Max | 1 |
| Heart rate zone | 1 |

For example, a display level of data related to activity information among the information on the fitness type user may be set as shown in Table 3.

TABLE 3

| Data | Display level |
|---|---|
| Time in motion | 2 |
| Steps | 2 |
| Activity calories | 2 |
| Readiness | 2 |
| Heart rate | 1 |
| Sweat loss | 1 |
| Oxygen saturation | 1 |
| Vo2Max | 1 |
| Heart rate zone | 1 |

For example, a display level of data related to group activity information among the information on the fitness type user may be set as shown in Table 4.

TABLE 4

| Data | Display level |
|---|---|
| Ranking | 2 |
| Participation Challenge ID | 2 |
| Total steps | 2 |
| Participation date | 2 |

Table 5, Table 6 and Table 7 may be examples of data included in information on the wellness type user.

For example, a display level of data related to sleep information among the information on the wellness type user may be set as shown in Table 5.

TABLE 5

| | Display |
|---|---|
| Data | level |
| sleep latency time | 2 |
| Wake-up time | 2 |
| Sleep time | 2 |
| Snoring time | 1 |
| Sleep state | 2 |

For example, a display level of data related to women's health information among the information on the wellness type user may be set as shown in Table 6.

TABLE 6

| Data | Display level |
|---|---|
| Menstrual cycle | 1 |
| Childbearing period | 1 |
| Ovulation day | 1 |

For example, a display level of data related to dietary intake information among the information on the wellness type user may be set as shown in Table 7.

TABLE 7

| Data | Display level |
|---|---|
| Intake calorie | 2 |
| Water intake | 2 |
| List of intake calorie | 2 |
| Food type | 2 |

Table 8, Table 9 and Table 10 may be examples of data included in information on the medical-type user.

For example, a display level of data related to biometric information among the information on the medical-type user may be set as shown in Table 8.

TABLE 8

| Data | Display level |
|---|---|
| Stress | 1 |
| Maximum heart rate | 1 |
| Minimum heart rate | 1 |
| Body temperature | 1 |
| Blood pressure | 1 |
| Blood sugar | 1 |

For example, a display level of data related to body information among the information on the medical-type user may be set as shown in Table 9.

TABLE 9

| Data | Display level |
|---|---|
| Age | 1 |
| Gender | 1 |
| Weight | 1 |
| Body composition | 1 |

For example, a display level of data related to dietary intake among the information on the medical-type user may be set as shown in Table 10.

TABLE 10

| Data | Display level |
|---|---|
| Intake calorie | 2 |
| Water intake | 2 |
| List of intake calorie | 2 |
| Food type | 2 |

The data included in Table 2 to Table 10 described above are examples and the disclosure is not limited thereto.

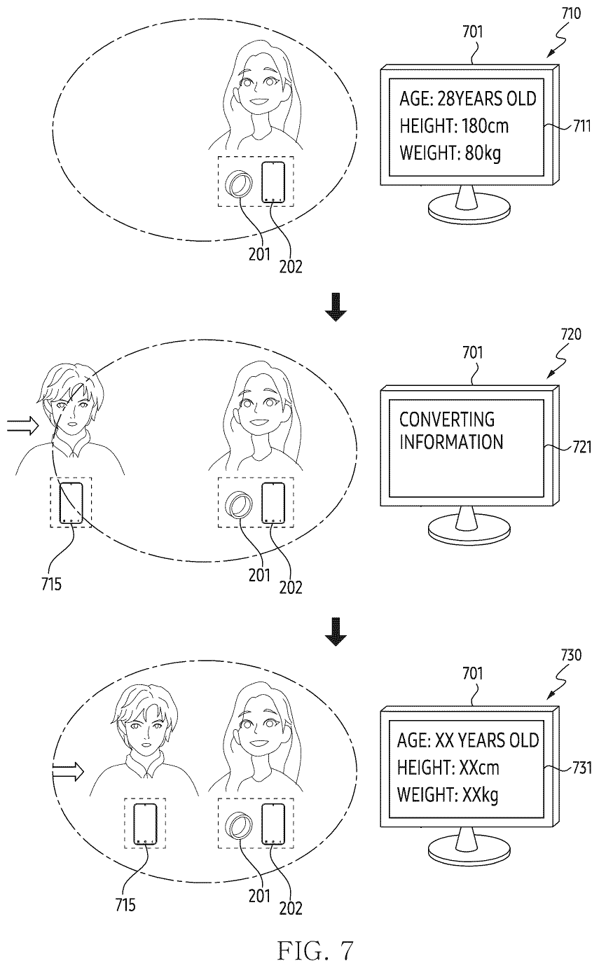

FIG. 7 is a diagram illustrating an example operation of a first electronic device, a second electronic device, and a first external electronic device, according to various embodiments. An operation of a first electronic device 201 described in FIG. 7 may be performed by a processor 310 of the first electronic device 201. An operation of a second electronic device 202 described in FIG. 7 may be performed by a processor 360 of the second electronic device 202. For convenience of description, it will be described that the operation of the first electronic device 201 is performed by the first electronic device 201. It will be described that the operation of the second electronic device 202 is performed by the second electronic device 202.

Referring to FIG. 7, in state 710, the first electronic device 201 may operate in a state connected with the second electronic device 202. For example, a first display level of information on a user obtained through a sensor 330 of the first electronic device 201 may be set through the second electronic device 202.

According to an embodiment, the first electronic device 201 may establish a connection with a first external electronic device 701. The first electronic device 201 may transmit a first signal for controlling to display a first screen 711 for representing information on the user through a display of the first external electronic device 701 to the first external electronic device 701.

For example, the second electronic device 202 may generate the first screen 711 for representing information on the user through the display of the first external electronic device 701 using a designated model. The second electronic device 202 may transmit information on the first screen 711 to the first electronic device 201. The first electronic device 201 may transmit the first signal to the first external electronic device 701, based on the information on the first screen 711 received from the second electronic device 202. The first external electronic device 701 may display the first screen 711 through the display of the first external electronic device 701 based on the first signal.

According to an embodiment, the first display level of information on the user may be set to a first value. A second display level related to the first external electronic device 701 may be set to the first value. Based on the first display level being less than or equal to the second display level, information on the user may be displayed through the display of the first external electronic device 701. For example, information on the user may be displayed on the first screen 711. The first screen 711 may be configured to represent information on the user. The first screen 711 may be configured to represent the user's age, the user's height, and the user's weight.

In state 720, the first electronic device 201 may identify the access of the second external electronic device 715 while the first screen 711 is displayed through the display of the first external electronic device 701. For example, the second external electronic device 715 may be owned by another user distinguished from the user. For example, the second external electronic device 715 may be used as a public device. For example, a display level of the second external electronic device 715 may be set to exceed a first value.

According to an embodiment, the first electronic device 201 may identify whether a distance between the second external electronic device 715 and the first electronic device 201 is within a designated distance. The first electronic device 201 may change the second display level related to the first external electronic device 701 based on that the distance between the second external electronic device 715 and the first electronic device 201 is within the designated distance. The first electronic device 201 may request information on a second screen 731 from the second electronic device 202, based on the change in the second display level related to the first external electronic device 701. The second electronic device 202 may generate the second screen 731 through the designated model, based on the request. The second electronic device 202 may transmit information on the second screen 731 to the first electronic device 201.

According to an embodiment, in response to identifying that the distance between the second external electronic device 715 and the first electronic device 201 is within the designated distance, the first electronic device 201 may transmit a third signal for controlling to display a third screen 721 representing that a screen displayed through the display of the first external electronic device 701 is being changed to the first external electronic device 701. For example, the first electronic device 201 may transmit the third signal for controlling to display the third screen 721 representing that the screen displayed through the display of the first external electronic device 701 is being changed to the first external electronic device 701, while the screen displayed through the display of the first external electronic device 701 is changed from the first screen 711 to the second screen 731.

For example, the first external electronic device 701 may change the screen displayed through the display of the first external electronic device 701 from the first screen 711 to the third screen 721 based on the third signal.

For example, the third screen 721 may not represent information on the user. While the second screen 731 is generated through the designated model of the second electronic device 202, the third screen 721 may be displayed through the display of the first external electronic device 701.

According to an embodiment, the first electronic device 201 may identify that the distance between the second external electronic device 715 and the first electronic device 201 is within the designated distance, based on the strength (e.g., RSSI) of a signal transmitted from the second external electronic device 715. According to an embodiment, the first electronic device 201 may identify that the distance between the second external electronic device 715 and the first electronic device 201 is within the designated distance, based on a designated software development kit (SDK).

In state 730, the first electronic device 201 may receive information on the second screen 731 from the second electronic device 202. The first electronic device 201 may transmit a second signal for controlling to change a screen displayed through the display of the first external electronic device 701 from the third screen 721 (or the first screen 711) to the second screen 731.

For example, the first external electronic device 701 may change the screen displayed through the display of the first external electronic device 701 from the third screen 721 to the second screen 731, based on the second signal.

For example, information on the user may be limited and displayed on the second screen 731. The second screen 731 may be configured to not represent specifically the user's age, the user's height, and the user's weight. For example, the second screen 731 may be configured to display information changed so that the user of the second external electronic device 715 may not recognize information on the user of the first electronic device 201. However, the disclosure is not limited thereto. For example, the second screen 731 may be configured to not display information on the user of the first electronic device 201. For example, the second screen 731 may be configured to display only part of the information on the user.

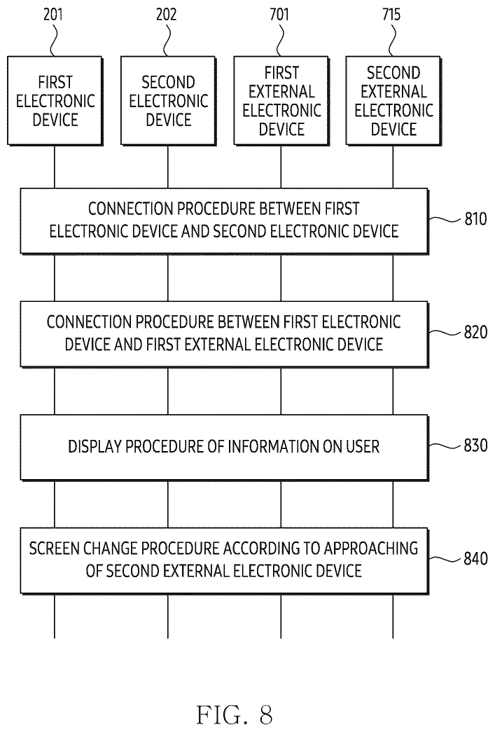

FIG. 8 is a signal flow diagram illustrating an example operation of a first electronic device, a second electronic device, a first external electronic device, and a second external electronic device, according to various embodiments. An operation of a first electronic device 201 described in FIG. 8 may be performed by a processor 310 of the first electronic device 201. An operation of a second electronic device 202 described in FIG. 8 may be performed by a processor 360 of the second electronic device 202. For convenience of description, it will be described that the operation of the first electronic device 201 is performed by the first electronic device 201. It will be described that the operation of the second electronic device 202 is performed by the second electronic device 202.

Referring to FIG. 8, in operation 810, a connection procedure between the first electronic device 201 and the second electronic device 202 may be performed. For example, the first electronic device 201 may establish a connection with the second electronic device 202. Based on establishing the connection with the second electronic device 202, the first electronic device 201 may identify a first display level of information on a user obtained through a sensor 330 of the first electronic device 201.

For example, a user interface for setting the first display level of information on the user may be displayed through a display 395 of the second electronic device 202. Based on the user interface displayed through the display 395 of the second electronic device 202, the first display level of information on the user may be set. The processor 310 may identify the first display level of user information set in the second electronic device 202.

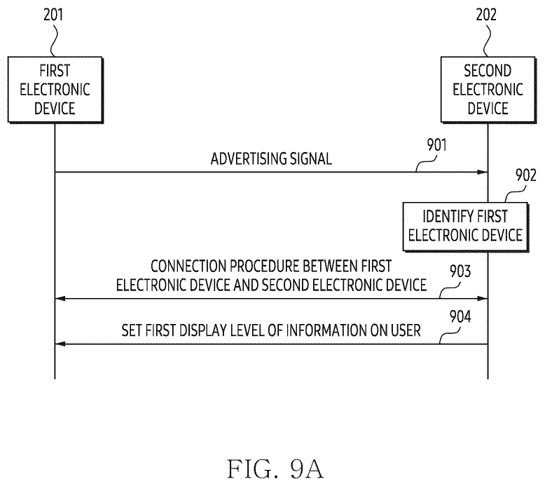

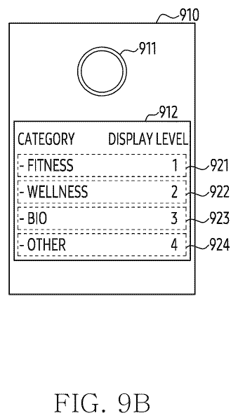

An example of a specific operation of operation 810 will be described later in FIGS. 9A and 9B.

In operation 820, a connection procedure between the first electronic device 201 and a first external electronic device 701 may be performed. The first electronic device 201 may establish a connection with the first external electronic device 701. The first electronic device 201 may identify a second display level related to the first external electronic device 701, based on the connection with the first external electronic device 701.

For example, the first electronic device 201 may identify at least one of information on a location of the first external electronic device 701, information on a space where the first external electronic device 701 is located, information on an account of the first external electronic device, information on an identifier of the first external electronic device, and/or information on a connection history of the first external electronic device. The first electronic device 201 may identify the second display level related to the first external electronic device 701, based on the at least one of information on a location of the first external electronic device 701, information on a space where the first external electronic device 701 is located, information on an account of the first external electronic device, information on an identifier of the first external electronic device, and/or information on a connection history of the first external electronic device.

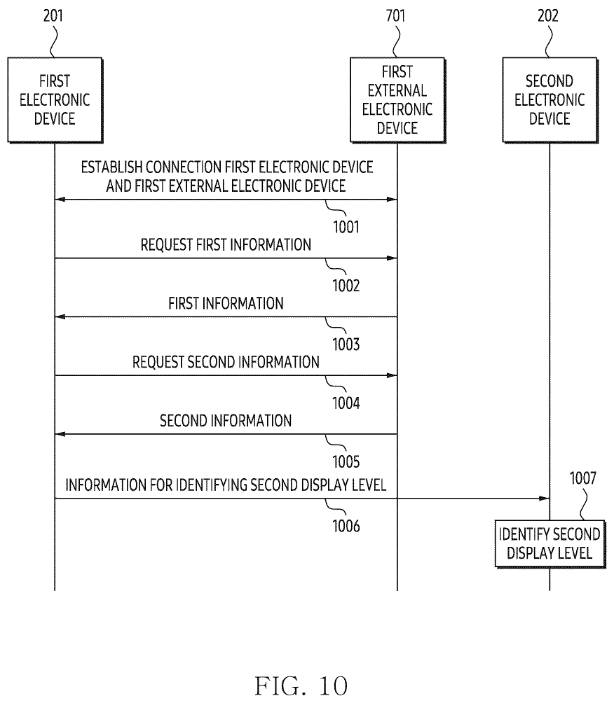

An example of a specific operation of operation 820 will be described in greater detail below with reference to FIG. 10.

In operation 830, a display procedure of information on the user may be performed. For example, based on the first display level and the second display level, the first electronic device 201 may transmit the first signal for controlling to display the first screen for representing information on the user through the display of the first external electronic device 701, to the first external electronic device 701.

For example, the first electronic device 201 may transmit a signal including the first display level and the second display level to the second electronic device 202. As an example, the signal may further include information on the user. As an example, the signal may include various information for generating a screen.

The second electronic device 202 may generate the first screen through a designated model, based on a signal including the first display level and the second display level. The second electronic device 202 may transmit information on the first screen to the first electronic device 201. The first electronic device 201 may transmit the first signal for controlling to display the first screen for representing information on the user through the display of the first external electronic device 701 to the first external electronic device 701, based on the information on the first screen.

For example, the second electronic device 202 may set the first display level and the second display level as an input value of the designated model. According to an embodiment, the second electronic device 202 may set various information in addition to the first display level and the second display level as the input value of the designated model. The various information may be obtained from the first electronic device 201 or may be obtained from the second electronic device 202.

As an example, at least one of profile information of the user, information on output setting (e.g., resolution, screen size, scan rate) of the display of the first external electronic device 701, information on a location of the first external electronic device 701, information on a space where the first external electronic device 701 is located, information on an account of the first external electronic device 701, information on an identifier of the first external electronic device 701, and/or information on a connection history of the first external electronic device 701 may be set as the input value of the designated model.

For example, the second electronic device 202 may generate (or obtain) the first screen, based on the output value of the designated model. The first screen may be configured to display information on the user, based on the first display level and the second display level. The first screen may represent at least a part of information on the user. As an example, the first screen may include multimedia content (e.g., image, text, video, user interface) for representing information on the user. As an example, the first screen may be configured to display information on a changed user based on the first display level and the second display level.

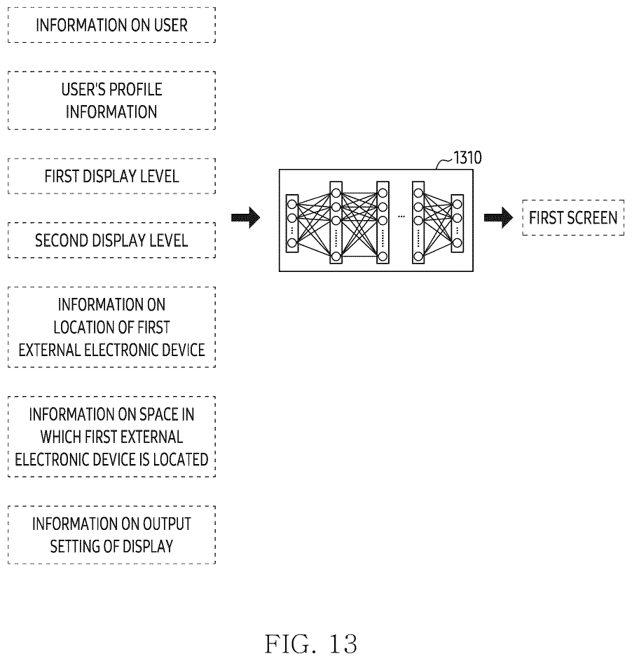

An example of a specific operation of generating a screen using a designated model in the second electronic device 202 will be described in greater detail below with reference to FIG. 13.

In operation 840, a screen change procedure according to the approach of the second external electronic device 715 may be performed. For example, based on identifying that the distance between the second external electronic device 715 and the first electronic device 201 is within a designated distance, the first electronic device 201 may transmit a second signal for controlling to change a screen displayed through the display of the first external electronic device 701 from the first screen to the second screen to the first external electronic device 701.

For example, the first electronic device 201 may change the second display level related to the first external electronic device 701, based on identifying that the distance between the second external electronic device 715 and the first electronic device 201 is within the designated distance. Based on changing the second display level related to the first external electronic device 701, the processor 310 may transmit the second signal for controlling to change the screen displayed through the display of the first external electronic device 701 from the first screen to the second screen 701, to the first external electronic device 701.

According to an embodiment, the first electronic device 201 may transmit information indicating that the distance between the second external electronic device 715 and the first electronic device 201 is within the designated distance to the second electronic device 202. The second electronic device 202 may change the second display level related to the first external electronic device 701. The second electronic device 202 may generate the second screen using the designated model, based on changing the second display level related to the first external electronic device 701. The second electronic device 202 may transmit information on the second screen to the first electronic device 201. The first electronic device 201 may transmit the second signal for controlling to change the screen displayed through the display of the first external electronic device 701 from the first screen to the second screen 701, to the first external electronic device 701.

The first external electronic device 701 may receive the second signal. Based on the second signal, the first external electronic device 701 may change the screen displayed through the display of the first external electronic device 701 from the first screen to the second screen.

FIG. 9A is a signal flow diagram illustrating example operations between a first electronic device and a second electronic device, according to various embodiments.

FIG. 9B is a diagram illustrating an example of a user interface for setting a first display level, according to various embodiments.

Referring to FIG. 9A, operations 901 to 904 may indicate a connection procedure between a first electronic device 201 and a second electronic device 202 and an operation for setting a first display level. Operations 901 to 904 may be related to operation 810 of FIG. 8.

In operation 901, the first electronic device 201 may transmit an advertising signal to the second electronic device 202. For example, the first electronic device 201 may transmit the advertising signal based on a broadcast method. The second electronic device 202 may receive the advertising signal transmitted from the first electronic device 201.

In operation 902, the second electronic device 202 may identify the first electronic device 201. For example, the second electronic device 202 may identify the first electronic device 201 based on the advertising signal. The second electronic device 202 may identify that the first electronic device 201 is located around the second electronic device 202 based on the advertising signal.

In operation 903, a connection procedure between the first electronic device 201 and the second electronic device 202 may be performed. The first electronic device 201 may establish a connection with the second electronic device 202. The second electronic device 202 may establish a connection with the first electronic device 201. The second electronic device 202 may register the first electronic device 201 as a device controllable in the second electronic device 202.

In operation 904, a first display level of information on a user may be set. For example, the first electronic device 201 may transmit a signal for requesting display of a screen for setting the first display level of information on the user to the second electronic device 202, based on the connection with the second electronic device 202. The second electronic device 202 may display a user interface for setting the first display level through a display 395 of the second electronic device 202, based on the received signal. Based on the user interface displayed through the display 395 of the second electronic device 202, the first display level of information on the user may be set. The first electronic device 201 may identify the first display level of the user information set at the second electronic device 202. The user interface for setting the first display level may correspond to a user interface 910 of FIG. 9B.

As described above, based on establishing the connection with the first electronic device 201, the second electronic device 202 may identify the first display level of information on the user (e.g., health information of the user) obtained based on a sensor (or at least one sensor) of the first electronic device 201. The second electronic device 202 may transmit information for indicating the first display level to the first electronic device 201.

Referring to FIG. 9B, the second electronic device 202 may display the user interface 910. The user interface 910 may include an object 911 representing a shape of the first electronic device 201. The user interface 910 may include an area 912 for setting the first display level of information on the user.

The area 912 may include areas for setting a first display level of information on a plurality of types of users. For example, the area 912 may include an area 921 for setting a first display level of information on a fitness type user. The area 912 may include an area 922 for setting a first display level of information on a wellness type user. The area 912 may include an area 923 for setting a first display level of information on a bio-type user. The area 912 may include an area 924 for setting a first display level of information on a user of other types.

Referring to FIG. 9A, the first display level of information on the user may be set in conjunction with an account service. According to an embodiment, a display level of information on other accounts related to an account of the user may also be set.

FIG. 10 is a signal flow diagram illustrating example operations between a first electronic device and a second electronic device, according to various embodiments.

Referring to FIG. 10, operations 1001 to 1007 may be related to an operation for identifying a second display level of a first external electronic device 701, based on a connection between a first electronic device 201 and the first external electronic device 701. Operations 1001 to 1007 may be related to operation 820 of FIG. 8.

In operation 1001, the first electronic device 201 and the first external electronic device 701 may establish a connection. For example, the first electronic device 201 may identify the first external electronic device 701 including a display. The first electronic device 201 may establish a connection with the first external electronic device 701, in order to display information on a user. For example, the first electronic device 201 may establish a connection with the first external electronic device 701 through various radio access technologies (RATs). For example, the first electronic device 201 may establish a connection with the first external electronic device 701, based on at least one of Bluetooth communication, wireless local area network (WLAN) communication, Zigbee communication, near field communication (NFC), ultra-wide band (UWB) communication, or ANT+ communication.

In operation 1002, the first electronic device 201 may request first information from the first external electronic device 701.

In operation 1003, the first external electronic device 701 may transmit the first information to the first electronic device 201. The first electronic device 201 may receive the first information from the first external electronic device 701. For example, the first information may include information on an identifier of the first external electronic device 701, information on device address of the first external electronic device 701, and/or encrypted information. For example, the encrypted information may include information on an account of the first external electronic device 701. For example, the first information may include information for identifying a second display level related to the first external electronic device 701.

In operation 1004, the first electronic device 201 may request second information from the first external electronic device 701. For example, the second information may include additional information for identifying the second display level related to the first external electronic device 701.

In operation 1005, the first external electronic device 701 may transmit the second information to the first electronic device 201. The first electronic device 201 may receive the second information from the first external electronic device 701. For example, the second information may include information on output setting (e.g., resolution, screen size, scan rate) of a display of the first external electronic device 701.

In operation 1006, the first electronic device 201 may transmit information for identifying the second display level related to the first external electronic device 701 to the second electronic device 202. The second electronic device 202 may receive the information for identifying the second display level related to the first external electronic device 701 from the first electronic device 201.

In operation 1007, the second electronic device 202 may identify the second display level related to the first external electronic device 701, based on the information for identifying the second display level related to the first external electronic device 701.

According to an embodiment, the second electronic device 202 may identify the second display level related to the first external electronic device 701, based on information on a location (or space) of the first electronic device 201 (or the user).

For example, a processor 360 of the second electronic device 202 may identify that the first electronic device 201 (or the user) is located in the user's home. The processor 360 may identify (or set) the second display level related to the first external electronic device 701 to the first value, based on identifying that the first electronic device 201 (or the user) is located in the user's home.

For example, the processor 360 may identify that the first electronic device 201 (or the user) is located in the living room in the user's home. The processor 360 may identify (or set) the second display level related to the first external electronic device 701 to a second value, based on identifying that the first electronic device 201 (or the user) is located in the living room in the user's home.

For example, the processor 360 may identify that the first electronic device 201 (or the user) is located in the private room in the user's home. The processor 360 may identify (or set) the second display level related to the first external electronic device 701 to the first value, based on identifying that the first electronic device 201 (or the user) is located in the private room in the user's home.

For example, the processor 360 may identify that the first electronic device 201 (or the user) is located in a fitness center. The processor 360 may identify (or set) the second display level related to the first external electronic device 701 to a fourth value, based on identifying that the first electronic device 201 (or the user) is located in the fitness center.

According to an embodiment, the second electronic device 202 may identify the second display level related to the first external electronic device 701, based on connectivity information on the first external electronic device 701. The connectivity information on the first external electronic device 701 may include at least one of information on the number of connections and/or contact information. The second electronic device 202 may identify a relationship between a user of the first external electronic device 701 and a user of the second electronic device 202 (or the first electronic device 201), based on at least one of contact information on the first external electronic device 701 or account information on the first external electronic device 701. For example, the second electronic device 202 may identify that the user of the first external electronic device 701 and the user of the second electronic device 202 (or the first electronic device 201) are in a family relationship with each other. For example, the second electronic device 202 may identify that the user of the first external electronic device 701 and the user of the second electronic device 202 (or the first electronic device 201) are in a friend relationship with each other. The second electronic device 202 may identify that the user of the first external electronic device 701 and the user of the second electronic device 202 (or the first electronic device 201) are the same.

The second electronic device 202 may identify the second display level based on a relationship between the user of the first external electronic device 701 and the user of the second electronic device 202 (or the first electronic device 201).

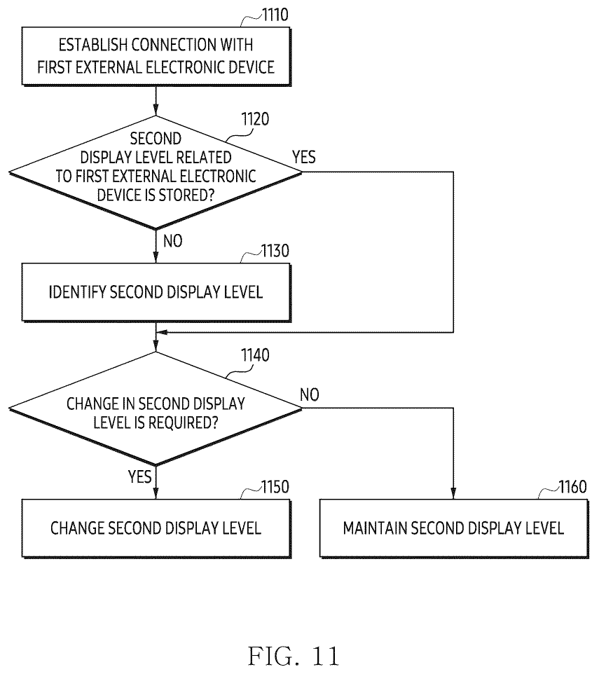

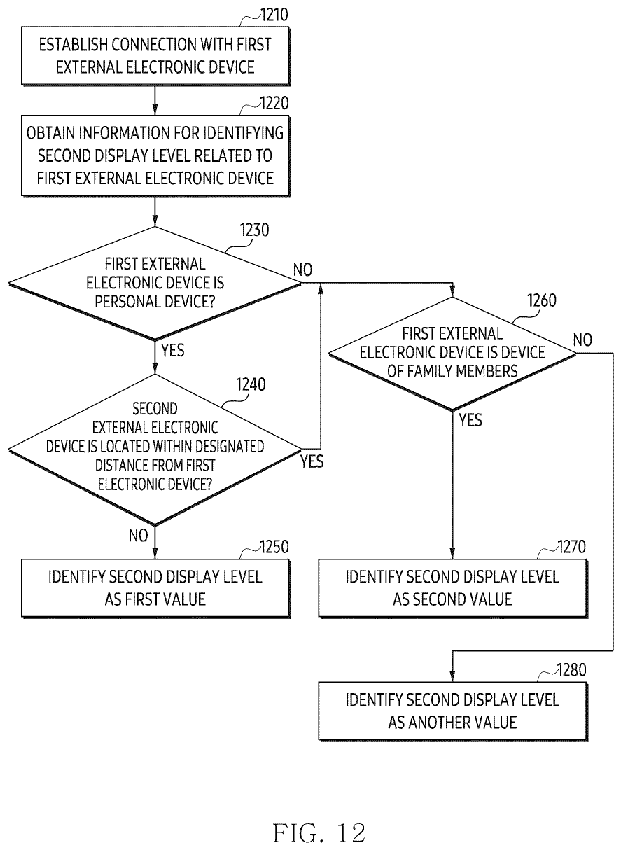

According to an embodiment, operation 1007 may be performed in the first electronic device 201. For example, the first electronic device 201 may identify the second display level related to the first external electronic device 701, based on the first information and the second information. For example, the first electronic device 201 may identify the second display level related to the first external electronic device 701, based on information for identifying the second display level related to the first external electronic device 701. An example of an operation of the first electronic device 201 when operation 1007 is performed at the first electronic device 201 will be described in greater detail below with reference to FIG. 11 or FIG. 12.

FIG. 11 is a flowchart illustrating an example operation of a first electronic device, according to various embodiments. In the following embodiment, each operation may be performed sequentially, but is not necessarily performed sequentially. For example, the sequence of each operation may be changed, and at least two operations may be performed in parallel.

For convenience of description, operation 1110 to operation 1160 are described as being performed at a first electronic device 201 (or a processor 310 of the first electronic device 201), but are not limited thereto. At least one operation of operation 1110 to operation 1160 may be performed at the second electronic device 202 (or the processor 360 of the second electronic device 202).

Referring to FIG. 11, in operation 1110, the processor 310 of the first electronic device 201 may establish a connection with a first external electronic device 701. Operation 1110 may correspond to operation 1001 of FIG. 10.

In operation 1120, the processor 310 may identify whether a second display level related to the first external electronic device 701 is stored in a memory 340.

According to an embodiment, when the second display level related to the first external electronic device 701 is stored in the memory 340, the processor 310 may identify the second display level stored in the memory 340. For example, the processor 310 may identify the second display level related to the first external electronic device 701 without receiving information for identifying the second display level related to the first external electronic device 701. The processor 310 may perform operation 1140 without performing operation 1130.

In operation 1130, when the second display level is not stored in the memory 340, the processor 310 may identify the second display level. For example, the processor 310 may identify the second display level based on identifying that the second display level is not stored in the memory 340.

For example, the processor 310 may receive (or obtain) information for identifying the second display level related to the first external electronic device 701 from the first external electronic device 701. The information for identifying the second display level related to the first external electronic device 701 may include the first information and the second information of FIG. 10. The processor 310 may identify the second display level related to the first external electronic device 701, based on the information for identifying the second display level related to the first external electronic device 701.

In operation 1140, the processor 310 may identify whether a change in the second display level is required.

For example, the second display level related to the first external electronic device 701 may be changed based on whether a distance between a second external electronic device 715 and the first electronic device 201 is within a designated distance.

For example, the second display level related to the first external electronic device 701 may be changed based on the number of devices within the designated distance from the first electronic device 201. For example, the second display level related to the first external electronic device 701 may be changed based on the number of signals identified at a location of the first electronic device 201.

For example, the second display level related to the first external electronic device 701 may be changed based on at least one of information on an account of the first external electronic device 701, information on an identifier of the first external electronic device 701, information on a location of the first external electronic device 701, or information on a connection history of the first external electronic device 701.

In operation 1150, when the second display level is required to be changed, the processor 310 may change the second display level of the first external electronic device 701.

For example, the processor 310 may identify that the distance between the second external electronic device 715 and the first electronic device 201 is within the designated distance. The processor 310 may change the second display level, based on identifying that the distance between the second external electronic device 715 and the first electronic device 201 is within the designated distance. For example, the processor 310 may increase the second display level, based on identifying that the distance between the second external electronic device 715 and the first electronic device 201 is within the designated distance. The processor 310 may restrict display of information on the user, based on increasing the second display level.

For example, the processor 310 may identify that the number of devices located within the designated distance from the second external electronic device 715 and the first electronic device 201 is greater than or equal to a designated number. The processor 310 may change the second display level, based on identifying that the number of devices located within the designated distance from the first electronic device 201 is greater than or equal to the designated number. As an example, the processor 310 may increase the second display level, based on identifying that the number of devices located within the designated distance from the first electronic device 201 is greater than or equal to the designated number. The processor 310 may restrict display of information on the user, based on increasing the second display level.

For example, the processor 310 may identify that the number of signals identified at a location of the first electronic device 201 is greater than or equal to a designated number. The processor 310 may change the second display level, based on identifying that the number of signals identified at the location of the first electronic device 201 is greater than or equal to the designated number. As an example, the processor 310 may increase the second display level, based on identifying that the number of signals identified at the location of the first electronic device 201 is greater than or equal to the designated number. The processor 310 may restrict display of information on the user, based on increasing the second display level.

For example, the processor 310 may identify that the at least one of information on an account of the first external electronic device 701, information on an identifier of the first external electronic device 701, information on a location of the first external electronic device 701, or information on a connection history of the first external electronic device 701 is changed. The processor 310 may change the second display level, based on identifying that the at least one of information on an account of the first external electronic device 701, information on an identifier of the first external electronic device 701, information on a location of the first external electronic device 701, or information on a connection history of the first external electronic device 701 is changed.

In operation 1160, when a change of the second display level is not required, the processor 310 may maintain the second display level of the first external electronic device 701. The processor 310 may maintain the second display level of the first external electronic device 701 based on identifying that a change of the second display level is not required.

FIG. 12 is a flowchart illustrating an example operation of a first electronic device, according to various embodiments. In the following embodiment, each operation may be performed sequentially, but is not necessarily performed sequentially. For example, the sequence of each operation may be changed, and at least two operations may be performed in parallel.

Operation 1210 to Operation 1280 may be performed to identify a second display level related to a first external electronic device 701. In FIG. 12, operation 1210 to operation 1280 are described as being performed by a first electronic device 201 (or a processor 310 of the first electronic device 201), but are not limited thereto. For example, operation 1210 to operation 1280 may be performed at a second electronic device 202 (or a processor 360 of the second electronic device 202).

Referring to FIG. 12, in operation 1210, the processor 310 may establish a connection with the first external electronic device 701. Operation 1210 may correspond to operation 1001 of FIG. 10.

In operation 1220, the processor 310 may obtain information for identifying the second display level related to the first external electronic device 701. For example, the processor 310 may receive (or obtain) information for identifying the second display level related to the first external electronic device 701 from the first external electronic device 701.

In operation 1230, the processor 310 may identify whether the first external electronic device 701 is a personal device. The processor 310 may identify whether the first external electronic device 701 is a personal device based on information on an account of the first external electronic device 701.

In operation 1240, when the first external electronic device 701 is a personal device, the processor 310 may identify whether the second external electronic device 715 is located within a designated distance from the first electronic device 201. For example, the processor 310 may identify whether the second external electronic device 715 is located within the designated distance from the first electronic device 201 based on identifying the first external electronic device 701 as a personal device.

In operation 1250, when the second external electronic device 715 is not located within the designated distance from the first electronic device 201, the processor 310 may identify (or set) the second display level as a first value (e.g., 1). The processor 310 may identify the second display level as the first value (e.g., 1), based on identifying that the second external electronic device 715 is not located within the designated distance from the first electronic device 201. When the second display level related to the first external electronic device 701 is identified (or set) as the first value, the first external electronic device 701 may display information on a user of the first display level set to a value greater than or equal to the first value.

In operation 1260, when the first external electronic device 701 is not a personal device or the second external electronic device 715 is located within the designated distance from the first electronic device 201, the processor 310 may identify whether the first external electronic device 701 is a device of family members. For example, the processor 310 may identify whether the first external electronic device 701 is a device of family members based on identifying that the first external electronic device 701 is not a personal device. For example, the processor 310 may identify whether the first external electronic device 701 is a device of family members based on identifying that the second external electronic device 715 is located within the designated distance from the first electronic device 201.

In operation 1270, when the first external electronic device 701 is a device of family members, the processor 310 may identify the second display level as a second value (e.g., 2). The processor 310 may identify the second display level as the second value, based on identifying that the first external electronic device 701 is a device of family members. When the second display level related to the first external electronic device 701 is identified (or set) as the second value, the first external electronic device 701 may display information on the user of the first display level set to a value greater than or equal to the second value.

In operation 1280, when the first external electronic device 701 is not a device of family members, the processor 310 may identify the second display level as another value (e.g., a third value or a fourth value) distinguished from the first value and the second value. The processor 310 may identify the second display level as another value distinguished from the first value and the second value, based on identifying that the first external electronic device 701 is not a device of family members.

In FIG. 12, an operation in which the second display level is identified based on a determination reference according to operation 1230, operation 1240, and operation 1260 is illustrated, but the disclosure is not limited thereto. In addition to the above-described operations, the second display level may be identified based on various information. For example, the second display level related to the first external electronic device 701 may be identified, based on at least one of information on a location of the first external electronic device 701, information on a space of the first external electronic device 701, information on an account of the first external electronic device 701, information on contact, and information on a relationship between the user of the first external electronic device 701 and the user of the first electronic device 201.

FIG. 13 is a diagram illustrating an example operation related to a designated model included in a second electronic device, according to various embodiments.

Referring to FIG. 13, a second electronic device 202 (or a processor 360 of the second electronic device 202) may include a designated model 1310. For example, the second electronic device 202 may include the designated model 1310 for generating (or obtaining) a screen for representing information on a user. The designated model may be configured based on a plurality of parameters.

For example, the designated model 1310 may be a model trained according to a supervised learning method based on an artificial intelligence algorithm or a model trained according to an unsupervised learning method. For example, a deep neural network (DNN), recurrent neural network (RNN), a bidirectional recurrent deep neural network (BRDNN), a generative adversarial network (GAN) may be used as a learning model, but are not limited thereto. For example, the designated model 1310 may be configured based on a generative artificial intelligence (AI) model.

For example, at least one of information on the user, information on a space in which the first external electronic device 701 is located, a first display level, a second display level, user's profile information, information on output setting (e.g., resolution, screen size, scan rate) of a display of the first external electronic device 701, and/or information on a location of the first external electronic device 701 may be set as an input value of the designated model 1310. Although not illustrated, information on an account of the first external electronic device 701, information on an identifier of the first external electronic device 701, and/or information on a connection history of the first external electronic device 701 may be set as an input value of the designated model 1310.

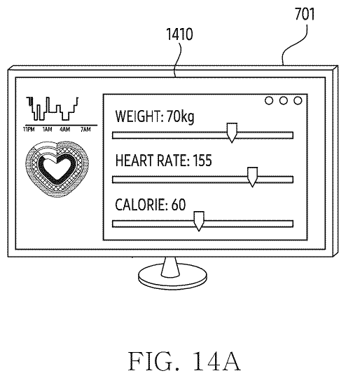

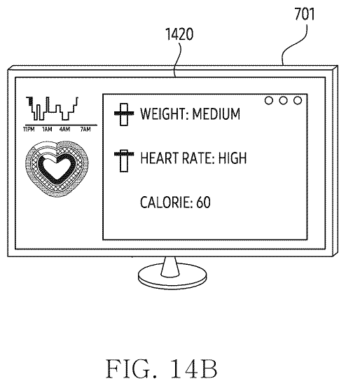

For example, the second electronic device 202 may generate a first screen (or a second screen) based on an output value of the designated model 1310. For example, the first screen (or the second screen) may include an image and/or text. The second electronic device 202 may generate the first screen (or the second screen) for representing information on the user through an image and/or text. For example, an example of the first screen generated through the output of the designated model 1310 may be illustrated in FIG. 14B.

In the above-described embodiment, an example in which the designated model is included in the second external electronic device is described, but is not limited thereto. The designated model may be included in a server. For example, the second electronic device 202 may transmit information for setting as an input value of a designated model to a server. The server may set information received from the second electronic device 202 as the input value of the designated model. Another device may identify information on the first screen based on an output value of the designated model. The server may transmit information on the first screen to the second electronic device 202. The second electronic device 202 may receive the information on the first screen from the server.

FIG. 14A is a diagram illustrating an example of a screen displayed at a first external electronic device in case that a designated model is not used, according to various embodiments.

FIG. 14B is a diagram illustrating an example of a screen displayed at a first external electronic device in case that a designated model is used, according to various embodiments.

Referring to FIGS. 14A and 14B, a second electronic device 202 may transmit information on a screen displayed at a first external electronic device 701 to a first electronic device 201. The first electronic device 201 may transmit a signal for controlling to display the screen to the first external electronic device 701.

For example, while a user of the first electronic device 201 is exercising, information on the user may be displayed through the first external electronic device 701.

In FIG. 14A, the second electronic device 202 may identify (or obtain) a screen 1410 without using a designated model. In the screen 1410, information on the user may be displayed in an unchanged state. As an example, in the screen 1410, specific values of the user's weight, the user's heart rate, and the user's calories burned may be displayed.

In FIG. 14B, the second electronic device 202 may generate a screen 1420 through the designated model. For example, display of the user's weight and the user's heart rate may be restricted, based on a first display level and a second display level. The screen 1420 generated through the designated model may display the user's weight and the user's heart rate with changes. For example, in the screen 1420, the user's weight may be displayed as ‘medium’. In the screen 1420, the user's heart rate may be displayed as ‘high’. In the screen 1420, the user's calories burned may remain substantially the same as in the screen 1410.

According to an embodiment, a layout of the designated model (or an output condition of the designated model) may be set. Based on the layout of the designated model, the screen 1420 may be generated. For example, when the weight or heart rate is set as an input value, the layout of the designated model may be set such that the weight or heart rate is changed to relative information within 10 characters. The layout of the designated model may be set such that items other than the weight or heart rate are maintained. The second electronic device 202 may generate the screen 1420 such that the weight or heart rate is changed to relative information and items other than the weight or heart rate are maintained.

According to an embodiment, the designated model may provide information on a method of displaying the screen 1420 based on information on output setting of the display of the first external electronic device 701.

For example, when the display level of the first external electronic device 701 is changed while the first screen (e.g., screen 1420) is displayed through the first external electronic device 701, the second electronic device 202 may generate a second screen in which the transparency of information displayed on the first screen is changed, using the designated model. For example, when the display level of the first external electronic device 701 is changed while the first screen is displayed through the first external electronic device 701, the second electronic device 202 may generate a second screen in which a size (e.g., a size of an object or text indicating information) of information displayed on the first screen is changed, using the designated model. As described above, the designated model may not only provide a screen in which information on the user is changed, but may also provide a screen in which an expression method of the information on the user is changed.

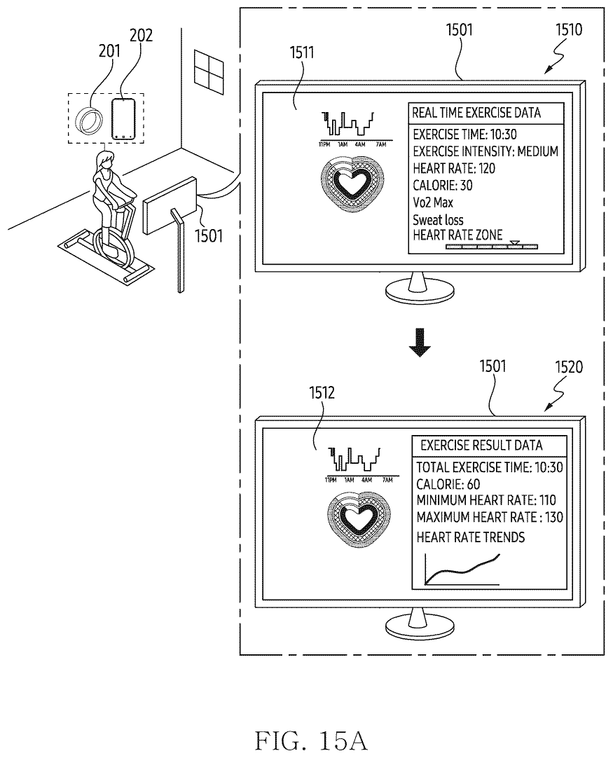

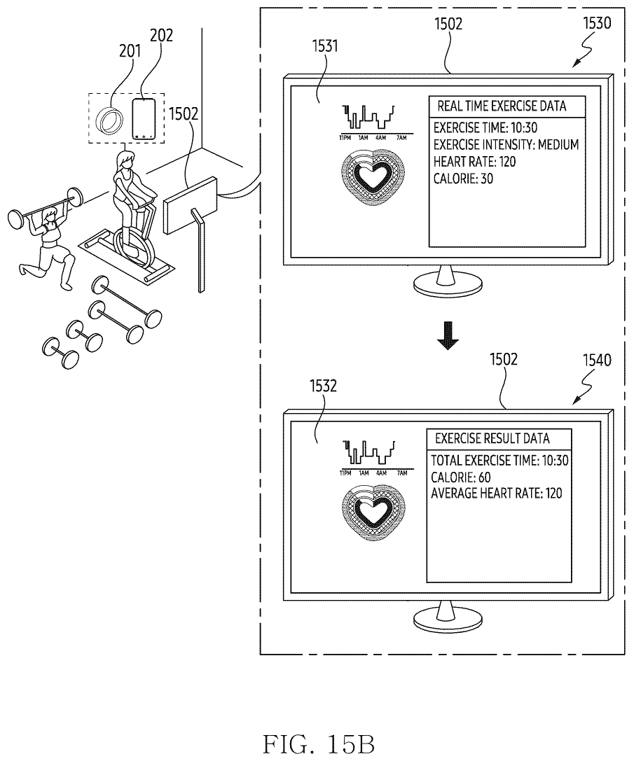

FIG. 15A is a diagram illustrating an example operation of a first electronic device, a second electronic device, and an external electronic device, according to various embodiments.

FIG. 15B is a diagram illustrating an example operation of a first electronic device, a second electronic device, and an external electronic device, according to various embodiments.

FIG. 15A is a diagram illustrating an example of screens displayed through an external electronic device 1501 when a user of a first electronic device 201 is located at the user's home. FIG. 15B is a diagram illustrating an example of screens displayed through the first external electronic device 1501 when the user of the first electronic device 201 is located at a public place (e.g., a fitness center).

Referring to FIG. 15A, the first electronic device 201 (or the user of the first electronic device 201) may be located at the user's home. The first electronic device 201 may operate in a state connected to the second electronic device 202. The first electronic device 201 may operate in a state connected to the external electronic device 1501 that is a user device. The first electronic device 201 may display exercise information of the user through the external electronic device 1501 while the user is exercising.

In state 1510, the first electronic device 201 may display the user's real-time exercise information through a display of the external electronic device 1501. For example, the first electronic device 201 may identify information on the user through a sensor 330. The information on the user may include information on exercise time, information on exercise intensity, information on heart rate, information on calories, information on Vo2Max, information on sweat loss, and/or information on heart rate zone.

The first electronic device 201 may identify a display level of each of the information on exercise time, the information on exercise intensity, the information on heart rate, and the information on calories as a fourth value. The first electronic device 201 may identify a display level of each of the information on Vo2Max, the information on sweat loss, and the information on heart rate zone as a first value.

The first electronic device 201 may set a display level of the external electronic device 1501 as a first value. For example, the first electronic device 201 may set the display level of the external electronic device 1501 as the first value, based on identifying the external electronic device 1501 as a device located in the user's home and registered to the user account.

The first electronic device 201 may transmit information on the user, information on a display level of the information on the user, and information on a display level related to the external electronic device 1501 to the second electronic device 202. Based on the information received from the first electronic device 201, the second electronic device 202 may generate a screen 1511 using a designated model. The second electronic device 202 may transmit information on the screen 1511 to the first electronic device 201. The first electronic device 201 may transmit a signal for controlling to display the screen 1511 to through a display of the external electronic device 1501 to the external electronic device 1501. The external electronic device 1501 may display the screen 1511 based on the received signal.

For example, since the display level of the external electronic device 1501 is set to the first value, all information on the user may be displayed on the screen 1511. As an example, all of information on exercise time, information on exercise intensity, information on heart rate, information on calories, information on Vo2Max, information on sweat loss, and/or information on heart rate zone may be displayed at the screen 1511.

In state 1520, the user may terminate the exercise. The first electronic device 201 may transmit information indicating that the user has terminated the exercise to the second electronic device 202. Based on identifying that the user has terminated the exercise, the second electronic device 202 may generate the screen 1512 using a designated model. The second electronic device 202 may transmit information on the screen 1512 to the first electronic device 201. The first electronic device 201 may transmit a signal for controlling to display the screen 1512 through the display of the first external electronic device 1501 to the first external electronic device 1501. The first external electronic device 1501 may display the screen 1512 based on the received signal.

For example, since the display level of the first external electronic device 1501 is set to the first value, all information on the user may be displayed in the screen 1512. As an example, all of information on total exercise time, information on calories, information on heart rate trends, maximum heart rate information, and minimum heart rate information may be displayed at the screen 1512.

Referring to FIG. 15B, the first electronic device 201 (or the user of the first electronic device 201) may be located at a public place (e.g., a fitness center). The first electronic device 201 may operate in a state connected to the second electronic device 202. The first electronic device 201 may operate in a state connected to a first external electronic device 1502 that is a public device. The first electronic device 201 may display the user's exercise information through the first external electronic device 1502 while the user is exercising.

In the state 1530, the first electronic device 201 may display the user's real time exercise information through a display of the external electronic device 1502. For example, the first electronic device 201 may identify information on the user through the sensor 330. The information on the user may include information on exercise time, information on exercise intensity, information on heart rate, information on calories, information on Vo2Max, information on sweat loss, and/or information on heart rate zone.

The first electronic device 201 may identify a display level of each of information on exercise time, information on exercise intensity, information on heart rate, and information on calories as the fourth value. The first electronic device 201 may identify a display level of each of information on Vo2Max, information on sweat loss, and information on heart rate zone as the first value.

The first electronic device 201 may set the display level of the external electronic device 1502 as the fourth value. For example, the first electronic device 201 may set the display level of the external electronic device 1502 as the fourth value, based on identifying that the external device 1502 is located at a public place (e.g., a fitness center) and the external electronic device is located within a designated distance from the first electronic device 201.

The first electronic device 201 may transmit information on the user, information on the display level of the information on the user, and information on the display level of the external electronic device 1502 to the second electronic device 202. Based on the information received from the first electronic device 201, the second electronic device 202 may generate a screen 1531 using a designated model. The second electronic device 202 may transmit information on the screen 1531 to the first electronic device 201. The first electronic device 201 may transmit a signal for controlling to display the screen 1531 through the display of the external electronic device 1502 to the external electronic device 1502. The external electronic device 1502 may display the screen 1531 based on the received signal.

For example, since the display level of the external electronic device 1502 is set to the fourth value, a part of the information on the user may be displayed in the screen 1531. As an example, information on exercise time, information on exercise intensity, information on heart rate, and/or information on calories may be displayed in the screen 1531. In the screen 1531, information on Vo2Max, information on sweat loss, and information on heart rate zone may not be displayed.

In state 1540, the user may terminate the exercise. The first electronic device 201 may transmit information indicating that the user has terminated the exercise to the second electronic device 202. Based on identifying that the user has terminated the exercise, the second electronic device 202 may generate a screen 1532 using a designated model. The second electronic device 202 may transmit information on the screen 1532 to the first electronic device 201. The first electronic device 201 may transmit a signal for controlling to display the screen 1532 through the display of the first external electronic device 1502 to the first external electronic device 1502. The first external electronic device 1502 may display the screen 1532 based on the received signal.

For example, since the display level of the first external electronic device 1502 is set to the fourth value, a part of the information on the user may be displayed in the screen 1532. As an example, unlike the screen 1512 of FIG. 15A, only information on total exercise time, information on calories, and information on average heart rate may be displayed on the screen 1532.

Referring to FIGS. 15A and 15B, according to a location of an external electronic device in which user information is displayed or whether the external electronic device is a personal device, the user information displayed on the external electronic device may be displayed differently.

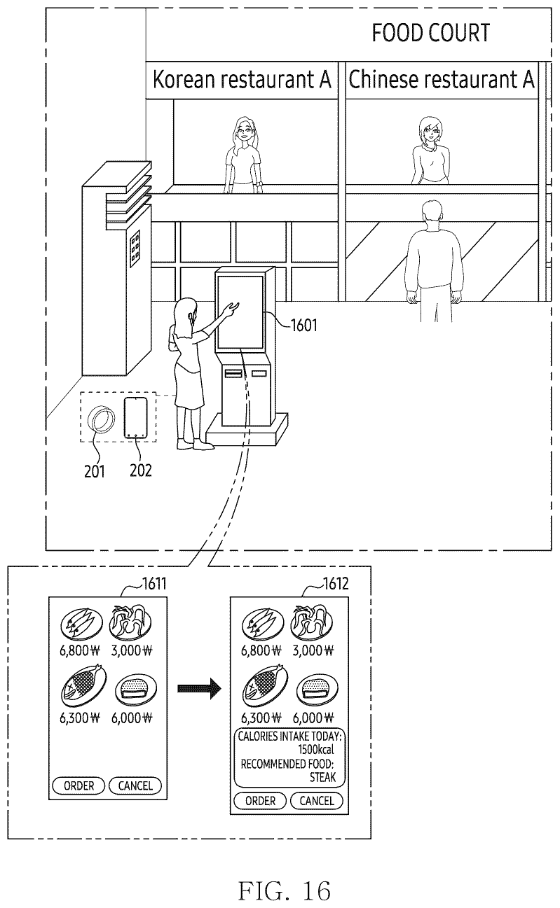

FIG. 16 is a diagram illustrating an example operation of a first electronic device, a second electronic device, and an external electronic device, according to various embodiments.

Referring to FIG. 16, a first electronic device 201 (or a user of the first electronic device 201) may be located in a restaurant (or a food court). The first electronic device 201 may operate in a state connected to a second electronic device 202. An external electronic device 1601 may be located in the restaurant (or food court). The external electronic device 1601 may be a kiosk for ordering food. The external electronic device 1601 may display a screen 1611 through a display. The screen 1611 may be configured to order food.

According to an embodiment, the first electronic device 201 may establish a connection with the external electronic device 1601. The first electronic device 201 may identify a display level related to the external electronic device 1601.

According to an embodiment, the first electronic device 201 may receive information to be displayed at the external electronic device 1601 from the second electronic device 202. For example, the first electronic device 201 may request the information to be displayed at the external electronic device 1601 from the second electronic device 202. The second electronic device 202 may transmit information on calories intake today and information on recommended food to the first electronic device 201, based on the display level related to the external electronic device 1601. The first electronic device 201 may receive the information on calories intake today and the information on recommended food from the second electronic device 202.

The first electronic device 201 may transmit the information on calories intake today and the information on recommended food to the external electronic device 1601. The external electronic device 1601 may display a screen 1612 based on the information on calories intake today and the information on recommended food. For example, the information on calories intake today and the information on recommended food may be displayed at the screen 1612. The user may select a menu based on the information on calories intake today and the information on recommended food displayed through the external electronic device 1601. According to an embodiment, the information on calories intake today and the information on recommended food may be displayed on a part of displaying area of a display of the external electronic device 1601. For example, the second electronic device 202 may identify a location in which the information on calories intake today and the information on recommended foods are displayed within the displaying area of the display of the external electronic device 1601.

According to an example embodiment, an electronic device (e.g., the first electronic device 201) may comprise: at least one sensor configured to detect health information of a user, a communication circuit configured to perform communication with an external device, memory including one or more storage mediums storing instructions, and at least one processor comprising processing circuitry. The instructions, when being executed by the at least one processor individually or collectively, may cause the electronic device to identify first level information (e.g., information on a first display level) of the health information of the user obtained based on the at least one sensor of the electronic device. The instructions, when being executed by the at least one processor individually or collectively, may cause the electronic device to identify second level information (e.g., information on a second display level) related to the first external electronic device, based on establishing a connection with a first external electronic device (e.g., the first external electronic device 701. The instructions, when being executed by the at least one processor individually or collectively, may cause the electronic device to transmit a first signal for controlling to display a first screen representing the health information of the user through a display of the first external electronic device, based on the first level information and the second level information.

According to an example embodiment, The instructions, when being executed by the at least one processor individually or collectively, may cause the electronic device to, while the health information of the user is displayed through the display of the first external electronic device, identify a distance between a second external electronic device (e.g., the second external electronic device 715) distinguished from the first external electronic device and the electronic device. The instructions, when being executed by the at least one processor individually or collectively, may cause the electronic device to change the second level information, based on identifying that the distance between the second external electronic device and the electronic device is within a designated distance. The instructions, when being executed by the at least one processor individually or collectively, may cause the electronic device to transmit a second signal for controlling to change a screen displayed through the display of the first external electronic device from the first screen to a second screen, based on changing the second level information.

According to an example embodiment, the instructions, when being executed by the at least one processor individually or collectively, may cause the electronic device to request the first level information to a third external electronic device (e.g., the second electronic device 202) based on establishing a connection with the third external electronic device. The instructions, when being executed by the at least one processor individually or collectively, may cause the electronic device to obtain, based on the request, the first level information from the third external electronic device.

According to an example embodiment, the first signal and the second signal may be set to be transmitted to the first external electronic device.

According to an example embodiment, the first screen may be generated through a designated model at the third external electronic device based on the first signal.

The second screen may be generated through the designated model at the third external electronic device based on the second signal.

According to an example embodiment, the first level information and the second level information may be set to an input value of the designated model. The first screen for representing the health information of the user may be set to an output value of the designated model.

According to an example embodiment, the health information of the user may be identified as one of a plurality of types. The instructions, when being executed by the at least one processor individually or collectively, may cause the electronic device to identify, based on the identified type, the first level information of the health information of the user.

According to an example embodiment, the instructions, when being executed by the at least one processor individually or collectively, may cause the electronic device to identify the second level information related to the first external electronic device based on at least one of information on an account of the first external electronic device, information on an identifier of the first external electronic device, information on a location of the first external electronic device, or information on a connection history of the first external electronic device.

According to an example embodiment, the instructions, when being executed by the at least one processor individually or collectively, may cause the electronic device to identify that the at least one of information on an account of the first external electronic device, information on an identifier of the first external electronic device, information on a location of the first external electronic device, or information on a connection history of the first external electronic device is changed. The instructions, when being executed by the at least one processor individually or collectively, may cause the electronic device to change the second level information related to the first external electronic device, based on identifying that the at least one of information on an account of the first external electronic device, information on an identifier of the first external electronic device, information on a location of the first external electronic device, or information on a connection history of the first external electronic device is changed.

According to an example embodiment, the instructions, when being executed by the at least one processor individually or collectively, may cause the electronic device to transmit a third signal for controlling to display a third screen for representing that the screen displayed through the display of the first external electronic device is being changed, while the screen displayed through the display of the first external electronic device is changed from the first screen to the second screen.

According to an example embodiment, the electronic device may further comprise: a ring-shaped housing comprising a first surface facing a part of a body of the user and a second surface opposite to the first surface. The at least one sensor, the communication circuit, and at least one processor may be disposed between the first surface and the second surface.

According to an example embodiment, a method for displaying a screen representing health information through an external device (e.g., the first electronic device 201) may comprise identifying first level information (e.g., information on the first display level) of health information of a user obtained based on at least one sensor of an electronic device. The method may comprise, based on establishing a connection with a first external electronic device (e.g., the first external electronic device 701), identifying second level information (e.g., information on the second display level) related to the first external electronic device. The method may comprise, based on the first level information and the second level information, transmitting a first signal for controlling to display a first screen for representing the health information of the user through a display of the first external electronic device.

According to an example embodiment, the method may comprise: while the first screen is displayed through the display of the first external electronic device, identifying a distance between a second external electronic device (e.g., the second external electronic device 715) distinguished from the first external electronic device and the electronic device. The method may comprise, based on identifying that the distance between the second external electronic device and the electronic device is within a designated distance, change the second level information. The method may comprise, based on changing the second level information, transmitting a second signal for controlling to change a screen displayed through the display of the first external electronic device from the first screen to a second screen.

According to an example embodiment, the method may comprise: requesting the first level information to a third external electronic device (e.g., the second electronic device 202), based on establishing a connection with the third external electronic device. The method may comprise obtaining, based on the request, the first level information from the third external electronic device.

According to an example embodiment, the first signal and the second signal may be set to be transmitted to the first external electronic device.

According to an example embodiment, the first screen may be generated through a designated model at the third external electronic device based on the first signal. The second screen may be generated through the designated model at the third external electronic device based on the second signal.

According to an example embodiment, the first level information and the second level information may be set to an input value of the designated model. The first screen for representing the health information of the user may be set to an output value of the designated model.

According to an example embodiment, the method may comprise changing the second level information to correspond to a third display level of the second external electronic device.

According to an example embodiment, the health information of the user may be identified as one of a plurality of types. The method may comprise identifying, based on the identified type, the first level information of the health information of the user.

According to an example embodiment, the method may comprise: identifying the second level information related to the first external electronic device based on at least one of information on an account of the first external electronic device, information on an identifier of the first external electronic device, information on a location of the first external electronic device, or information on a connection history of the first external electronic device.

According to an example embodiment, the method may comprise: transmitting a third signal for displaying a third screen for representing that the screen displayed through the display of the first external electronic device is being changed, while the screen displayed through the display of the first external electronic device is changed from the first screen to the second screen.

According to an example embodiment, an electronic device (e.g., the second electronic device 202) may comprise: a communication circuit configured to perform communication with an external device, memory including one or more storage mediums storing instructions, and at least one processor comprising processing circuitry. The instructions, when being executed by the at least one processor individually or collectively, may cause the electronic device to set first level information of health information of a user obtained based on at least one sensor of the first external electronic device and second level information, based on establishing a connection with a first external electronic device (e.g., the first electronic device 201). The instructions, when being executed by the at least one processor individually or collectively, may cause the electronic device to transmit the first level information and the second level information to the first external electronic device.

According to an example embodiment, the instructions, when being executed by the at least one processor individually or collectively, may cause the electronic device to transmit the second level information to the first external electronic device, based on a connection between the first external electronic device and a second external electronic device (e.g., the first external electronic device 701).

According to an example embodiment, The instructions, when being executed by the at least one processor individually or collectively, may cause the electronic device to obtain information on the second external electronic device. The instructions, when being executed by the at least one processor individually or collectively, may cause the electronic device to set, based on the information on the second external electronic device, the second level information.

According to an example embodiment, The instructions, when being executed by the at least one processor individually or collectively, may cause the electronic device to obtain, based on a designated model stored in the memory, a first screen for representing the health information of the user. The instructions, when being executed by the at least one processor individually or collectively, may cause the electronic device to transmit information on the first screen to the first external electronic device.

According to an example embodiment, the electronic device may comprise a display. The instructions, when being executed by the at least one processor individually or collectively, may cause the electronic device to display, based on the first level information, a screen representing the health information through the display.

According to an example embodiment, the instructions, when being executed by the at least one processor individually or collectively, may cause the electronic device to display a user interface to set the first level information through the display. The instructions, when being executed by the at least one processor individually or collectively, may cause the electronic device to identify, based on the user interface, an input to set the first level information; set, based on the input, the first level information. The instructions, when being executed by the at least one processor individually or collectively, may cause the electronic device to transmit the first level information to the first external electronic device.

According to the various example embodiments, a user may display information obtained from a first electronic device that does not include a display through a display of a first external electronic device. Since at least part of the information on the user may be displayed based on a first display level of information on the user and a second display level of the first external electronic device, the privacy of the user may be protected. For example, when a second external electronic device is located within a designated distance from the first electronic device, information displayed through the display of the first external electronic device may be changed. Accordingly, the privacy of the user may be strengthened.

The processor may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of or all possible combinations of the items enumerated together in a corresponding one of the phrases. As used herein, such terms as “1st” and “2nd,” or “first” and “second” may be used to simply distinguish a corresponding component from another, and docs not limit the components in other aspect (e.g., importance or order). It is to be understood that if an element (e.g., a first element) is referred to, with or without the term “operatively” or “communicatively”, as “coupled with,” or “connected with” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between a case in which data is semi-permanently stored in the storage medium and a case in which the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

No claim element is to be construed under the provisions of 35 U.S.C. § 112, sixth paragraph, unless the element is expressly recited using the phrase “means for” or “means.”

What is claimed is:

1. An electronic device comprising:
at least one sensor configured to detect health information of a user;
a communication circuit configured to perform communication with an external device;
memory including one or more storage mediums storing instructions; and
at least one processor comprising processing circuitry,
wherein the instructions, when being executed by the at least one processor individually or collectively, cause the electronic device to:
identify first level information of the health information of the user obtained based on the at least one sensor of the electronic device,
based on establishing a connection with a first external electronic device, identify second level information related to the first external electronic device, and
based on the first level information and the second level information, transmit a first signal for controlling to display a first screen representing the health information of the user through a display of the first external electronic device.

2. The electronic device of claim 1, wherein the instructions, when being executed by the at least one processor individually or collectively, cause the electronic device to:
based on the health information of the user being displayed through the display of the first external electronic device, identify a distance between a second external electronic device distinguished from the first external electronic device and the electronic device,
based on identifying that the distance between the second external electronic device and the electronic device is within a designated distance, change the second level information, and
based on changing the second level information, transmit a second signal for controlling to change a screen displayed through the display of the first external electronic device from the first screen to a second screen.

3. The electronic device of claim 2, wherein the instructions, when being executed by the at least one processor individually or collectively, cause the electronic device to:
request the first level information to a third external electronic device based on establishing a connection with the third external electronic device, and
obtain, based on the request, the first level information from the third external electronic device.

4. The electronic device of claim 3, wherein the first signal and the second signal are set to be transmitted to the first external electronic device.

5. The electronic device of claim 3, wherein the first screen is generated through a designated model at the third external electronic device based on the first signal, and
wherein the second screen is generated through the designated model at the third external electronic device based on the second signal.

6. The electronic device of claim 5, wherein the first level information and the second level information are set to an input value of the designated model, and
wherein the first screen representing the health information of the user is set to an output value of the designated model.

7. The electronic device of claim 1, wherein the health information of the user is identified as one of a plurality of types, and
wherein the instructions, when being executed by the at least one processor individually or collectively, cause the electronic device to identify, based on the identified type, the first level information of the health information of the user.

8. The electronic device of claim 1, wherein the instructions, when being executed by the at least one processor individually or collectively, cause the electronic device to identify the second level information related to the first external electronic device based on at least one of information on an account of the first external electronic device, information on an identifier of the first external electronic device, information on a location of the first external electronic device, or information on a connection history of the first external electronic device.

9. The electronic device of claim 8, wherein the instructions, when being executed by the at least one processor individually or collectively, cause the electronic device to:
identify that the at least one of information on an account of the first external electronic device, information on an identifier of the first external electronic device, information on a location of the first external electronic device, or information on a connection history of the first external electronic device is changed, and
based on identifying that the at least one of information on an account of the first external electronic device, information on an identifier of the first external electronic device, information on a location of the first external electronic device, or information on a connection history of the first external electronic device is changed, change the second level information related to the first external electronic device.

10. The electronic device of claim 2, wherein the instructions, when being executed by the at least one processor individually or collectively, cause the electronic device to transmit a third signal to display a third screen representing that the screen displayed through the display of the first external electronic device is being changed, based on the screen displayed through the display of the first external electronic device being changed from the first screen to the second screen.

11. The electronic device of claim 1, further comprising a ring-shaped housing comprising a first surface facing a part of a body of the user and a second surface opposite to the first surface,
wherein the at least one sensor, the communication circuit, and the at least one processor are disposed between the first surface and the second surface.

12. A method for displaying a screen representing health information through an external device comprising:
identifying first level information of health information of a user obtained based on at least one sensor of an electronic device,
based on establishing a connection with a first external electronic device, identifying second level information related to the first external electronic device, and
based on the first level information and the second level information, transmitting a first signal for controlling to display a first screen representing the health information of the user through a display of the first external electronic device.

13. The method of claim 12, wherein the method further comprises:
based on the first screen being displayed through the display of the first external electronic device, identifying a distance between a second external electronic device distinguished from the first external electronic device and the electronic device,
based on identifying that the distance between the second external electronic device and the electronic device is within a designated distance, change the second level information, and
based on changing the second level information, transmitting a second signal for controlling to change a screen displayed through the display of the first external electronic device from the first screen to a second screen.

14. The method of claim 12, further comprising:
requesting the first level information to a third external electronic device based on establishing a connection with the third external electronic device, and
obtaining, based on the request, the first level information from the third external electronic device.

15. The method of claim 14, wherein the first signal and the second signal are set to be transmitted to the first external electronic device.

16. The method of claim 14, wherein the first screen is generated through a designated model at the third external electronic device based on the first signal, and
wherein the second screen is generated through the designated model at the third external electronic device based on the second signal.

17. The method of claim 16, wherein the first level information and the second level information are set to an input value of the designated model, and
wherein the first screen representing the health information of the user is set to an output value of the designated model.

18. The method of claim 12, wherein the method further comprises changing the second level information to correspond to a third display level of the second external electronic device.

19. The method of claim 12, wherein the health information of the user is identified as one of a plurality of types, and
wherein the method further comprises identifying, based on the identified type, the first level information of the health information of the user.

20. The method of claim 12, further comprising identifying the second level information related to the first external electronic device based on at least one of information on an account of the first external electronic device, information on an identifier of the first external electronic device, information on a location of the first external electronic device, or information on a connection history of the first external electronic device.
